(19) United States
(12) Patent Application Publication
(10) Pub. No.: US 2007/0043062 A1
(43) Pub. Date: Feb. 22, 2007

(54) Isoxazoles as peptide deformylase inhibitors

(75) Inventors: Patrizia Cali, Copenhagen S (DK); Anders Hjelmencrantz, Malmo (SE); Lars Naerum, Hellerup (DK)

Correspondence Address: EDWARDS & ANGELL, LLP, P.O. BOX 55874, BOSTON, MA, 02205, US

(73) Assignee: Arpida A/S, Frederiksberg C (DK)

(21) Appl. No.: 10/571,361

(22) PCT Filed: Sep. 10, 2004
(86) PCT No.: PCT/DK04/00604

§ 371 Date: Mar. 10, 2006

Related U.S. Application Data
(60) Provisional application No. 60/503,104, filed on Sep. 15, 2003.

(30) Foreign Application Priority Data
Sep. 12, 2003 (DK) PA 2003 01323

(51) Int. Cl. A61K 31/506; A61K 31/42; C07D 413/02

(52) U.S. Cl. 514/256; 514/378; 544/333; 548/248

(57) ABSTRACT

Isoxazole compounds of formula (I) and pharmaceutically acceptable salts or esters thereof are peptide deformylase inhibitors useful in the treatment or prevention of infections and other disease in which peptide deformylases are involved, especially in the treatment of bacterial and parasitic infections, for example infections fully or partly caused by microorganisms belonging to Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, Helicobacter, Campylobacter, Legionella or Neisseria.

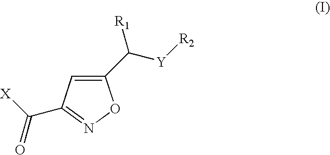

The present invention relates to novel enzyme inhibitors, more specifically to inhibitors of peptide deformylase useful in the treatment or prevention of infections and other diseases in which peptide deformylases are involved, especially in the treatment of bacterial and parasitic infections. More specifically the invention relates to isoxazoles capable of inhibiting bacterial peptide deformylase, also known as PDF.

### BACKGROUND OF THE INVENTION

Peptide deformylase (EC 3.4.1.88), also known as PDF, is an enzyme that catalyzes the deformylation of formyl-L-methionyl peptides. PDF removes the formyl group from the N-terminal Met of newly synthesized proteins, i.e. catalyzes the conversion of formyl-L-methionyl peptide to methionyl peptide (Adams and Capecchi, 1966; Adams, 1968).

PDF is essential to bacteria, and bacterial peptide deformylase (PDF) is now widely recognised as an attractive target for antibacterial chemotherapy (Giglione et al., 2000; Giglione and Meinnel, 2001; Pei 2001; Yuan et al., 2001; Clements et al., 2002). Deformylation is a crucial step in bacterial protein biosynthesis and PDF is an essential ingredient in bacterial growth, with the gene encoding PDF present in all sequenced pathogenic bacterial genomes.

Novel antibacterial compounds are urgently needed due to the growing resistance exhibited by both Gram-negative and Gram-positive bacteria and other microorganisms. Traditional antibiotics have targeted pathways unique to bacterial replication and maintenance. However, new pathways are not being targeted in a manner that outpaces the growth of bacterial resistance. Thus, novel compounds and strategies are greatly needed that can be used to eradicate resistant bacteria.

### SUMMARY OF THE INVENTION

The present invention relates to compounds of the general formula (I) 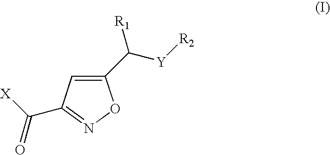
wherein R₁, R₂, X and Y are as defined in the detailed part of this description.

It is contemplated that the compounds of the invention are useful for the treatment of infections caused by bacteria or parasites. It is especially contemplated that the compounds of the present invention are useful for the treatment of infections fully or partly caused by Gram-positive or Gram-negative bacteria such as Escherichia coli and Staphylococcus aureus or by a parasite such as Plasmodium falciparum.

It is an object of the invention to provide novel compounds having pharmacological activity as inhibitors of PDF.

Further objects will become apparent from the following description.

### DETAILED DESCRIPTION OF THE INVENTION

Definitions

The term “peptide deformylase” or “PDF” as used herein is intended to mean peptide deformylase (EC 3.4.1.88), also known as PDF, which catalyzes the conversion of the N-terminal formyl-L-methionyl peptide to methionyl peptide in newly synthesized proteins.

The term “treatment” is defined as the management and care of a patient for the purpose of combating the disease, condition, or disorder and includes the administration of a compound of the present invention to prevent the onset of the symptoms or the complications, or alleviating the symptoms or the complications, or eliminating the disease, condition, or disorder.

As used herein, alone or in combination, the term “C₁₋₆ alkyl” denotes a straight or branched, saturated hydrocarbon chain having from one to six carbon atoms. C₁₋₆ alkyl groups include, but are not limited to, methyl, ethyl, n-propyl, iso-propyl, n-butyl, isobutyl, sec-butyl, tert-butyl, n-pentyl, iso-pentyl, 2-methylbutyl, 3-methylbutyl, n-hexyl, iso-hexyl, 4-methylpentyl, neopentyl, 2,2-dimethylpropyl and the like.

As used herein, alone or in combination, the term “C₂₋₆ alkenyl” denotes a straight or branched, unsaturated hydrocarbon chain having from two to six carbon atoms and at least one double bond. C₂₋₆ alkenyl groups include, but are not limited to, vinyl, 1-propenyl, allyl, iso-propenyl, n-butenyl, n-pentenyl, n-hexenyl and the like.

The term “C₁₋₆ alkoxy” in the present context designates a group O—C₁₋₆ alkyl used alone or in combination, wherein C₁₋₆ alkyl is as defined above. Examples of linear alkoxy groups are methoxy, ethoxy, propoxy, butoxy, pentoxy and hexoxy. Examples of branched alkoxy are iso-propoxy, sec-butoxy, tert-butoxy, iso-pentoxy and iso-hexoxy. Examples of cyclic alkoxy are cyclopropyloxy, cyclobutyloxy, cyclopentyloxy and cyclohexyloxy.

The term “aminocarbonylC₁₋₆ alkyl” in the present context designates a group NH—C(O)—C₁₋₆ alkyl used alone or in combination, wherein C₁₋₆ alkyl is as defined above. Examples of aminocarbonylC₁₋₆ alkyl groups include, but are not limited to, aminocarbonylmethyl and aminocarbonylethyl.

The term “C₃₋₁₀ cycloalkyl” as used herein denotes a radical of one or more saturated mono-, bi-, tri- or spirocyclic hydrocarbon having from three to ten carbon atoms. Examples include, but are not limited to, cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₇ heterocycloalkyl” as used herein denotes a radical of a totally saturated heterocycle like a cyclic hydrocarbon containing one or more heteroatoms selected from nitrogen, oxygen and sulphur independently in the cycle. Examples of heterocycles include, but are not limited to, pyrrolidine (1-pyrrolidine, 2-pyrrolidine, 3-pyrrolidine, 4-pyrrolidine, 5-pyrrolidine), pyrazolidine (1-pyrazolidine, 2-pyrazolidine, 3-pyrazolidine, 4-pyrazolidine, 5-pyrazolidine), imidazolidine (1-imidazolidine, 2-imidazolidine, 3-imidazolidine, 4-imidazolidine, 5-imidazolidine), thiazolidine (2-thiazolidine, 3-thiazolidine, 4-thiazolidine, 5-thiazolidine), piperidine (1-piperidine, 2-piperidine, 3-piperidine, 4-piperidine, 5-piperidine, 6-piperidine), piperazine (1-piperazine, 2-piperazine, 3-piperazine, 4-piperazine, 5-piperazine, 6-piperazine), morpholine (2-morpholine, 3-morpholine, 4-morpholine, 5-morpholine, 6-morpholine), thiomorpholine (2-thiomorpholine, 3-thiomorpholine, 4-thiomorpholine, 5-thiomorpholine, 6-thiomorpholine), 1,2-oxathiolane (3-(1,2-oxathiolane), 4-(1,2-oxathiolane), 5-(1,2-oxathiolane)), 1,3-dioxolane (2-(1,3-dioxolane), 3-(1,3-dioxolane), 4-(1,3-dioxolane)), tetrahydropyrane (2-tetrahydropyrane, 3-tetrahydropyrane, 4-tetrahydropyrane, 5-tetrahydropyrane, 6-tetrahydropyrane), hexahydropyradizine, (1-(hexahydropyradizine), 2-(hexahydropyradizine), 3-(hexahydropyradizine), 4-(hexahydropyradizine), 5-(hexahydropyradizine), 6-(hexahydropyradizine)).

The term “aryl” as used herein is intended to include carbocyclic aromatic ring systems. Aryl is also intended to include the partially hydrogenated derivatives of the carbocyclic systems enumerated below.

The term “heteroaryl” as used herein includes heterocyclic aromatic ring systems containing one or more heteroatoms selected from nitrogen, oxygen and sulphur such as furyl, thienyl, pyrrolyl, and is also intended to include the partially hydrogenated derivatives of the heterocyclic systems enumerated below.

Examples include, but are not limited to, phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), phenanthrenyl, fluorenyl, pentalenyl, azulenyl, biphenylenyl, thiophenyl (1-thienyl, 2-thienyl), furyl (1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl(thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), isoquinolyl (1-isoquinolyl, 3-isoquinolyl, 4-isoquinolyl, 5-isoquinolyl, 6-isoquinolyl, 7-isoquinolyl, 8-isoquinolyl), quinolyl (2-quinolyl, 3-quinolyl, 4-quinolyl, 5-quinolyl, 6-quinolyl, 7-quinolyl, 8-quinolyl), benzo[b]furanyl (2-benzo[b]furanyl, 3-benzo[b]furanyl, 4-benzo[b]furanyl, 5-benzo[b]furanyl, 6-benzol[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzol[b]thiophenyl, 7-benzo[b]thiophenyl), 2,3-dihydrobenzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl (1-indolyl, 2-indolyl, 3-indolyl, 4-indolyl, 5-indolyl, 6-indolyl, 7-indolyl), indazolyl (1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

The term “C₁₋₆ alkylaryl” as used herein refers to an aryl group as defined above attached through a C₁₋₄ alkyl group as defined above having one, two, three, four, five or six carbon atoms.

The term “C₁₋₆ alkylheteroaryl” as used herein refers to a heteroaryl group as defined above attached through a C₁₋₆ alkyl group as defined above having one, two, three, four, five or six carbon atoms.

“Halogen” designates an atom selected from the group consisting of F, Cl, Br and I.

The terms “unsubstituted” or “substituted” as used herein means that the groups in question are optionally unsubstituted or substituted with one or more, for example one, two, three or four of the substituents specified. Preferably, the groups in question are substituted with one or more, for example one, two, three or four substituents independently of each other selected from halogen, C₁₋₄ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano. When the groups in question are substituted with more than one substituent, the substituents may be the same or different.

Certain of the above defined terms may occur more than once in the structural formula, and upon such occurrence each term shall be defined independently of the other.

As used herein, the phrase “a functional group, which can be converted to hydrogen in vivo” is intended to include any group which upon administering the present compounds to the subjects in need thereof can be converted to hydrogen e.g. enzymatically or by the acidic environment in the stomach. Non-limiting examples of such groups are acyl, carbamoyl, monoalkylated carbamoyl, dialkylated carbamoyl, alkoxycarbonyl, alkoxyalkyl groups and the like such as C₁₋₆ alkylcarbonyl, aroyl, C₁₋₆ alkylcarbamoyl, di-C₁₋₆ alkyl-alkylcarbamoyl, C₁₋₆ alkoxycarbonyl and C₁₋₆ alkoxy-C₁₋₆ alkyl.

As used herein, the phrase “diseases and disorders related to peptide deformylase” is intended to include any disease or disorder in which an effect, preferably an inhibiting effect, on peptide deformylase is beneficial, especially on the bacterial peptide deformylase.

The term “IC₅₀” as used herein denotes the concentration required for 50% inhibition of PDF in a binding assay.

The Compounds

The present invention relates of compounds of the general formula (I) 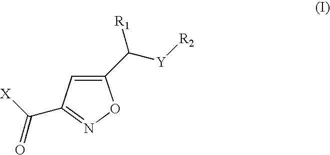
or a pharmaceutically acceptable salt or ester thereof, wherein
X is selected from hydroxy, C₁₋₆ alkoxy, and —NH—OH;
Y is selected from S, SO, SO₂ and O;
R₁ is selected from hydrogen, C₁₋₆ alkyl, C₂₋₆ alkenyl, C₃₋₁₀ cycloalkyl, C₃₋₇ heterocycloalkyl, an unsubstituted or substituted C₁₋₆ alkylaryl group, an unsubstituted or substituted C₁₋₆ alkylheteroaryl group, an unsubstituted or substituted aryl group, an unsubstituted or substituted heteroaryl group, wherein a substituted group in connection with R₁ is substituted with one, two, three or four substituents independently selected from the group consisting of halogen, C₁₋₆ alkyl and C₁₋₆ alkoxy; and R₂ is selected from C₁₋₆ alkyl, C₃₋₁₀ cycloalkyl, C₃₋₇ heterocycloalkyl, an unsubstituted or substituted aryl group, an unsubstituted or substituted heteroaryl group, an unsubstituted or substituted C₁₋₆ alkylaryl group, or an unsubstituted or substituted C₁₋₆ alkylheteroaryl group, wherein a substituted group in connection with R₂ is substituted with one, two, three or four substituents independently selected from the group consisting of halogen, C₁₋₆ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano;
with the proviso that when X is hydroxy or ethoxy, Y is S, SO, or SO₂ and R₁ is hydrogen, R₂ cannot be methyl;
with the proviso that when X is methoxy or ethoxy, Y is S and R₁ is hydrogen, R₂ cannot be a 4-halogen-pyridazin-3-on;
with the proviso that when X is methoxy, Y is S and R₁ is methyl, R₂ cannot be a 4-halogen-pyridazin-3-on.

In a preferred embodiment of the invention, X is hydroxy, methoxy, ethoxy, propoxy or —NH—OH.

In another preferred embodiment of the invention, Y is S, SO or SO₂, more preferably S (sulfur).

In yet another preferred embodiment of the invention, R₁ is hydrogen, C₁₋₆ alkyl, or C₃₋₁₀ cycloalkyl; more preferably hydrogen, methyl, ethyl, n-propyl, isopropyl, n-butyl, sec. butyl, isobutyl, tert-butyl, pentyl, cyclopentyl or cyclohexyl; or R₁ is an unsubstituted or substituted aryl group, an unsubstituted or substituted heteroaryl group, or R₁ is selected from an unsubstituted or substituted C₁₋₆ alkylaryl group and an unsubstituted or substituted C₁₋₆ alkylheteroaryl group.

R₂ is selected from an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group.

R₂ may also be an unsubstituted or substituted aryl group, or an unsubstituted or substituted phenyl group.

In preferred embodiments, R₂ is a substituted phenyl group, wherein a substituted phenyl is substituted with one, two, three or four substituents independently selected from methyl, ethyl, n-propyl, butyl, isopropyl, isobutyl, sec-butyl, tert-butyl, fluoro, chloro, bromo, iodo, methoxy, trifluoromethyl, trifluoromethoxy, aminocarbonylmethyl and thiophenyl. In specific embodiments R₂ is a substituted phenyl group, substituted with one, two, three or four substituents independently selected from chloro, bromo, trifluoromethyl or trifluoromethoxy.

Examples of a suitable R₂ group is an unsubstituted or substituted group, wherein the group is selected from biphenyl, diphenyl, naphtyl, benzothiophenylmethyl, thiophenyl-pyrimidinyl, pyridyl, quinolyl, pyrimidinyl, pyrazinyl, pyridazinyl, pyrazolyl, imidazolyl, triazolyl, tetrazolyl, oxazolyl, isoxazolyl, thiazolyl, isothiazolyl, thiophenyl, furanyl, thiadiazolyl and oxadiazolyl.

In another embodiment, R₂ is selected from C₁₋₆ alkyl, C₃₋₁₀ cycloalkyl and C₃₋₇ heterocycloalkyl.

Preferred compounds of the invention are:
5-(4-toluenesulfanylmethyl)-isoxazole-3-carboxylic acid hydroxyamide;
5-(4-chloro-phenylsulfanylmethyl)-isoxazole-3-carboxylic acid hydroxyamide;
5-(3-chloro-phenylsulfanylmethyl)-isoxazole-3-carboxylic acid hydroxyamide;
5-(2-chloro-phenylsulfanylmethyl)-isoxazole-3-carboxylic acid hydroxyamide;
5-(3,4-dichloro-phenylsulfanylmethyl)-isoxazole-3-carboxylic acid hydroxyamide;
5-(2-bromo-phenylsulfanylmethyl)-isoxazole-3-carboxylic acid hydroxyamide;
5-(3-bromo-phenylsulfanylmethyl)-isoxazole-3-carboxylic acid hydroxyamide;
5-(2-isopropyl-phenylsulfanylmethyl)-isoxazole-3-carboxylic acid hydroxyamide;
5-(2,4,6-trimethyl-phenylsulfanylmethyl)-isoxazole-3-carboxylic acid hydroxyamide;
5-(3-methoxy-phenylsulfanylmethyl)-isoxazole-3-carbocylic acid hydroxyamide;
5-(4-fluoro-phenylsulfanylmethyl)-isoxazole-3-carboxylic acid hydroxyamide;
5-(4-trifluoromethyl-phenylsulfanylmethyl)-isoxazole-3-carboxylic acid hydroxyamide;
5-(2-trifluoromethyl-phenylsulfanylmethyl)-isoxazole-3-carboxylic acid hydroxyamide;
5-(2-trifluoromethoxy-phenylsulfanylmethyl)-isoxazole-3-carboxylic acid hydroxyamide;
5-(3-trifluoromethyl-benzylsulfanylmethyl)-isoxazole-3-carboxylic acid hydroxyamide;
5-(4-toluenesulfanylmethyl)-isoxazole-3-carboxylic acid;
5-(4-chloro-phenylsulfanylmethyl)-isoxazole-3-carboxylic acid;
5-(3-chloro-phenylsulfanylmethyl)-isoxazole-3-carboxylic acid;
5-(2-isopropyl-phenylsulfanylmethyl)-isoxazole-3-carboxylic acid;
5-(2,4,6-trimethyl-phenylsulfanylmethyl)-isoxazole-3-carboxylic acid;
5-(4-methoxy-phenylsulfanylmethyl)-isoxazole-3-carboxylic acid;
5-(3-methoxy-phenylsulfanylmethyl)-isoxazole-3-carboxylic acid;
5-(4-fluoro-phenylsulfanylmethyl)-isoxazole-3-carboxylic acid;
5-(4-trifluoromethyl-phenylsulfanylmethyl)-isoxazole-3-carboxylic acid;
5-(2-trifluoromethyl-phenylsulfanylmethyl)-isoxazole-3-carboxylic acid;
5-(2-trifluoromethoxy-phenylsulfanylmethyl)-isoxazol e-3-carboxylic acid;
5-(3-trifluoromethyl-benzylsulfanylmethyl)-isoxazole-3-carboxylic acid;
5-(4-toluenesulfonylmethyl)-isoxazole-3-carboxylic acid hydroxyamide;
5-(4-chloro-benzenesulfonylmethyl)-isoxazole-3-carboxylic acid hydroxyamide;
5-(3-chloro-benzenesulfonylmethyl)-isoxazole-3-carboxylic acid hydroxyamide;
5-(2-isopropyl-benzenesulfonylmethyl)-isoxazole-3-carboxylic acid hydroxyamide;
5-(3-methoxy-benzenesulfonylmethyl)-isoxazole-3-carboxylic acid hydroxyamide;
5-(4-toluenesulfinylmethyl)-isoxazole-3-carboxylic acid hydroxyamide;
5-[1-(4-toluenesulfonyl)-ethyl]-isoxazole-3-carboxylic acid hydroxyamide;
5-[1-(4-chloro-benzenesulfonyl)-ethyl]-isoxazole-3-carboxylic acid hydroxyamide;
5-[1-(3-methoxy-benzenesulfonyl)-propyl]-isoxazole-3-carboxylic acid hydroxyamide;
5-[1-(2-isopropyl-benzenesulfonyl)-propyl]-isoxazole-3-carboxylic acid hydroxyamide;
5-(benzothiophen-2-ylmethylsulfanylmethyl)-isoxazole-3-carboxylic acid hydroxyamide;
5-(4-thiophen-2-yl-pyrimidin-2-ylsulfanylmethyl)-isoxazole-3-carboxylic acid hydroxyamide;
5-(benzothiophen-2-ylmethylsulfanylmethyl)-isoxazole-3-carboxylic acid; and
5-(4-thiophen-2-yl-pyrimidin-2-ylsulfanylmethyl)-isoxazole-3-carboxylic acid.

The compounds of the invention may exist as geometric isomers or optical isomers or stereoisomers as well as tautomers. Accordingly, the invention includes all geometric isomers and tautomers including mixtures and racemic mixtures of these and a pharmaceutically acceptable salt thereof, especially all R- and S-isomers.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the Formula I. Prodrugs are any covalently bonded compounds, which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form. Conventional procedures for the selection and preparation of suitable prodrug derivatives are described, for example, in “Design of Prodrugs”, ed. H. Bundgaard, Elsevier, (1985).

The invention also encompasses active metabolites of the present compounds.

The present invention includes all complexes of the compounds of this invention.

In a preferred embodiment of this invention, the compounds of Formula I exhibit an IC₅₀ value of less than 500 μM, preferably less than 100 μM, more preferably less than 50 μM, even more preferably less than 1 μM, especially less than 500 nM, particularly less than 100 nM, when subjected to a bacterial PDF assay.

A compound according to the present invention can be used in medicine, in particular as a protease inhibitor such as a peptide deformylase inhibitor.

A compound according to the invention can also be used in the treatment, prophylaxis and/or diagnosis of bacterial infections fully or partly caused by an organism belonging to any of the genera Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, Helicobacter, Campylobacter, Legionella and Neisseria.

Synthetic Method of Preparation

The compounds of the present invention may be prepared by the methods set forth in the schemes A-E below.

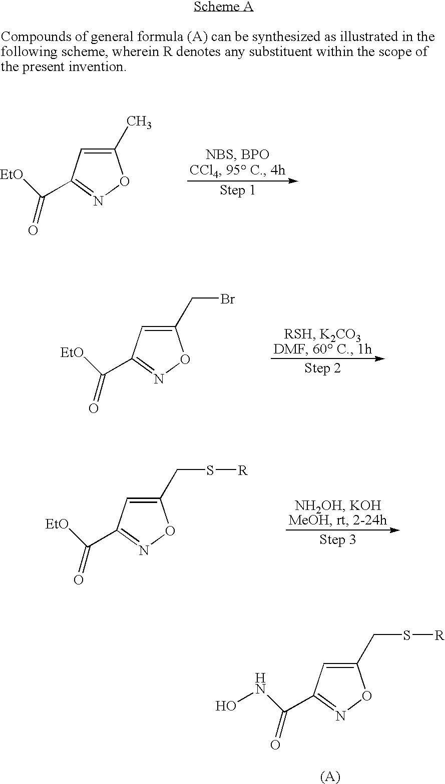

The general synthetic scheme involves the initial radical bromination of ethyl-5-methylisoxazole-3-carboxylate using N-bromosuccinimide and benzoylperoxide as radical initiator. The reaction of the intermediate benzylic bromide with an array of thiols, for example aromatic, heteroaromatic and benzylic thiols, afford the desired thioether. Reaction of the ethyl ester with a methanolic solution of hydroxylamine hydrochloride afforded the desired hydroxamic acid.

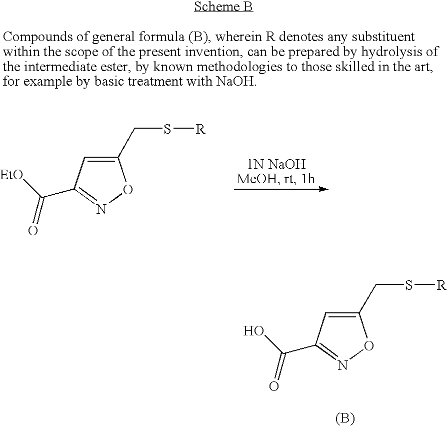

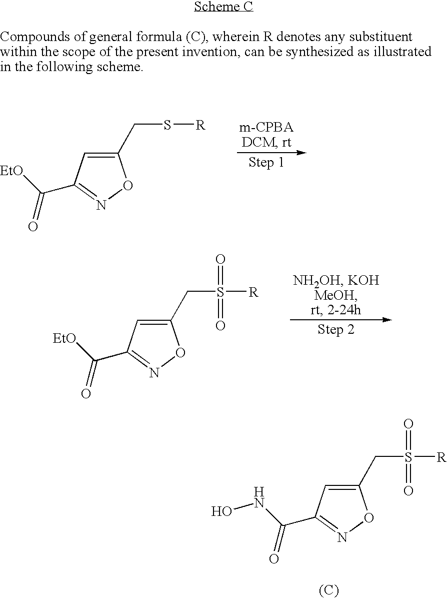

After oxidation of the intermediate thioether to sulfone (for example using m-chloroperbenzoic acid), the desired hydroxamic acid is obtained by reaction with a methanolic solution of hydroxylamine hydrochloride.

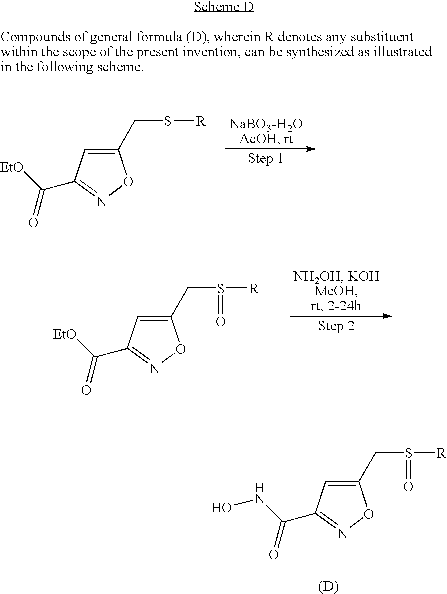

Selective mono-oxidation of the intermediate thioether can be achieved for example by using NaBO₃.H₂O as oxidant, and monitoring the conversion by HPLC-MS (Mass Spectrometry). The desired hydroxamic acid is then obtained by reaction with a methanolic solution of hydroxylamine hydrochloride.

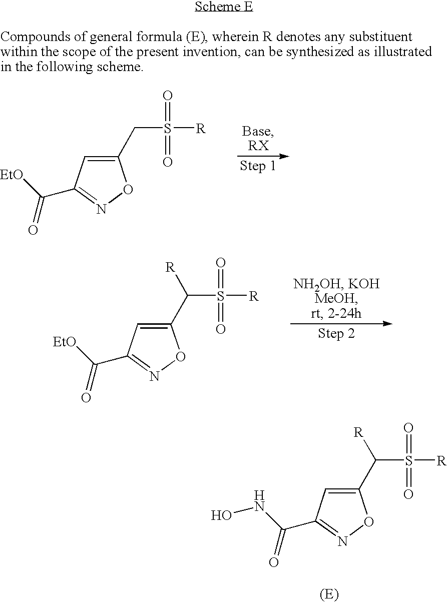

Alkylation of the benzylic position can be achieved by treating the intermediate previously obtained (Step 1, Scheme C) with a suitable base (for example NaH), followed by addition of an electrophile (for example alkyl/benzyl halides). The desired hydroxamic acid is then obtained by reaction with a methanolic solution of hydroxylamine hydrochloride.

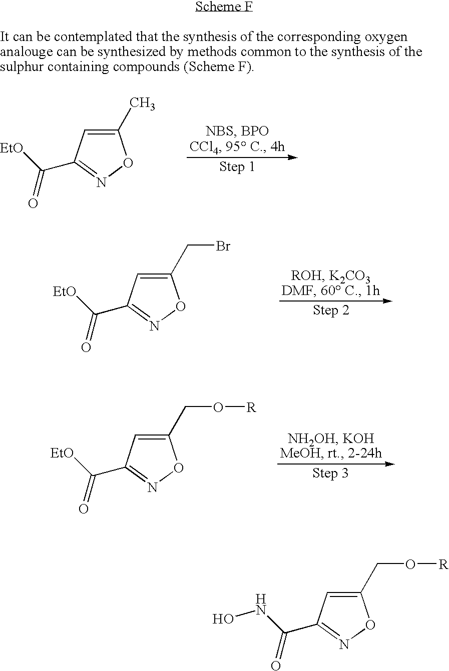

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions, which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

Pharmaceutical Compositions

In one aspect of this invention, there is provided a pharmaceutical composition comprising, as an active ingredient, a compound of the present invention together with a pharmaceutically acceptable carrier or diluent. This composition may be in unit dosage form and may comprise from about 1 μg to about 1000 mg such as, e.g., from about 10 μg to about 500 mg, from about 0.05 to about 100 mg or from about 0.1 to about 50 mg, of the compound of the invention or a pharmaceutically acceptable salt or ester thereof. The composition of the invention may be used for oral, nasal, transdermal, pulmonal or parenteral administration. It is contemplated that the pharmaceutical composition of the invention is useful for treatment of bacterial and/or parasitic infections.

The compounds of the invention may be administered alone or in combination with pharmaceutically acceptable carriers, diluents or excipients, in either single or multiple doses. Accordingly, the compounds of Formula I may be used in the manufacture of a medicament. The pharmaceutical compositions according to the invention may be formulated with pharmaceutically acceptable carriers or diluents as well as any other known adjuvants and excipients in accordance with conventional techniques, for example such as those disclosed in Remington (Gennaro and Gennaro (1995)).

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 50 mg/kg body weight per day, preferably from about 0.01 to about 30 mg/kg body weight per day, and more preferred from about 0.05 to about 20 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from about 0.05 to about 500 mg, preferably from about 0.05 to about 100 mg, more preferably from about 0.1 to about 50 mg, and more preferred from about 0.5 mg to about 20 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the Formula (I) contains a free base such salts are prepared in a conventional manner by treating a solution or suspension of a free base of the Formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the Formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the Formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tableted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain:

Core:

| | |
|---|---|
| Active compound (free compound or salt) | 5.0 mg |
| Lactosum Ph. Eur. | 67.8 mg |
| Cellulose, microcryst. (Avicel) | 31.4 mg |
| Amberlite | 1.0 mg |
| Magnesii stearas | q.s. |

Coating:

| | |
|---|---|
| Hydroxypropyl methylcellulose approx. | 9 mg |
| Acylated monoglyceride approx. | 0.9 mg |

If desired, the pharmaceutical composition of the invention may comprise the compound of the Formula (I) in combination with further pharmacologically active substances such as those described in the foregoing.

Use of the Invention

The compounds of formula I according to the invention are useful in medicine. Moreover, the compounds of formula I 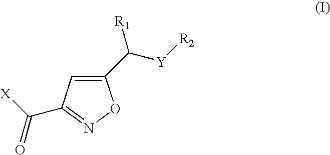
or a pharmaceutically acceptable salt or ester thereof,
wherein
X is selected from hydroxy, C₁₋₆ alkoxy, and —NH—OH;
Y is selected from S, SO, SO₂ and O;
R₁ is selected from hydrogen, C₁₋₆ alkyl, C₂₋₆ alkenyl, C₃₋₁₀ cycloalkyl, C₃₋₇ heterocycloalkyl, an unsubstituted or substituted C₁₋₆ alkylaryl group, an unsubstituted or substituted C₁₋₆ alkylheteroaryl group, an unsubstituted or substituted aryl group, an unsubstituted or substituted heteroaryl group, wherein a substituted group in connection with R₁ is substituted with one, two, three or four substituents independently selected from the group consisting of halogen, C₁₋₆ alkyl and C₁₋₆ alkoxy; and
R₂ is selected from C₁₋₆ alkyl, C₃₋₁₀ cycloalkyl, C₃₋₇ heterocycloalkyl, an unsubstituted or substituted aryl group, an unsubstituted or substituted heteroaryl group, an unsubstituted or substituted C₁₋₆ alkylaryl group, or an unsubstituted or substituted C₁₋₆ alkylheteroaryl group, wherein a substituted group in connection with R₂ is substituted with one, two, three or four substituents independently selected from the group consisting of halogen, C₁₋₆ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano;
may be used for the preparation of a medicament for treatment of bacterial infections.

The scope of the compounds claimed takes into account a few previously described compounds that fall within the definition of formula I, but which have been excluded by a disclaimer. However, these compounds are contemplated to have therapeutic activity against bacterial infections and are therefore included in the aspects of the invention related to a method for treatment of ailments or use of compounds according to formula I.

The compounds of formula I (with or without disclaimer) may be used as protease inhibitors, particularly as inhibitors of metallo proteases, more particularly as inhibitors of peptide deformylase, even more particularly as inhibitors of bacterial peptide deformylase. The present invention provides useful compositions and formulations of said compounds, including pharmaceutical compositions and formulations of said compounds.

The compounds of the present invention may be especially useful for the treatment or prevention of diseases caused by a variety of bacterial or prokaryotic organisms. Examples include Gram-positive and Gram-negative aerobic and anaerobic bacteria such as, Staphylococci, for example S. aureus and S. epidermidis; Enterococci, for example E. faecium and E. faecalis; Streptococci, for example S. pneumoniae; Haemophilus, for example H. influenzae; Moraxella, for example M. catarrhalis; Escherichia, for example E. coli; Mycobacteria, for example M. tuberculosis and M. ranae; Mycoplasma, for example M. pneumoniae; Pseudomonas, for example P. aeruginosa; intercellular microbes, for example Chlamydia and Rickettsiae. Other examples include Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae. It is further contemplated that the compounds of the present invention are useful for the treatment of parasitic infections, for example infections caused by Plasmodium falciparum and the like.

Accordingly, in one aspect the present invention relates to a method for the treatment of ailments, the method comprising administering to a subject in need thereof an effective amount of a compound or a composition of this invention. It is contemplated that an effective amount of a compound or a composition of this invention corresponds to an amount of active ingredient, i.e. active compound or a pharmaceutically acceptable salt or ester thereof, in the range of from about 1 μg to about 1000 mg such as, e.g., from about 10 μg to about 500 mg, from about 0.05 to about 100 mg or from about 0.1 to about 50 mg per day.

In yet another aspect, the present invention relates to use of a compound of this invention for the preparation of a medicament, preferably a medicament for the treatment of infections caused by Gram-positive or Gram-negative aerobic or anaerobic bacteria, or by parasites.

In a preferred embodiment of the invention, there is provided a medicament for the treatment of infections caused by Staphylococci, Enterococci, Streptococci, Haemophilus, Moraxella, Escherichia, Mycobacteria, Mycoplasma, Pseudomonas, Chlamydia, Rickettsiae, Klebsiella, Shigella, Salmonella, Bordetella, Clostridia, Heliobacter, Campylobacter, Legionella and Neisseria, preferably caused by Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylon, Campylobacter jejuni, Legionella pneumophila and Neisseria gonorrhoeae.

It is further contemplated that the compounds of the present invention are useful for the treatment of parasitic infections, for example infections caused by Plasmodium falciparum and the like.

An intravenous infusion of the compound in 5% dextrose in water or normal saline, or a similar formulation with suitable excipients, is most effective, although an intramuscular bone injection is also useful. Typically, the parenteral dose will be about 0.01 to about 100 mg/kg; preferably between 0.1 and 20 mg/kg, in a manner to maintain the concentration of drug in the plasma at a concentration effective to inhibit PDF. The compounds may be administered one to four times daily at a level to achieve a total daily dose of about 0.4 to about 400 mg/kg/day. The precise amount of an inventive compound which is therapeutically effective, and the route by which such compound is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

The compounds of the present invention fully or partly inhibit bacterial PDF, and are thus useful for the treatment and/or prevention of a wide variety of conditions and disorders in which inhibition of PDF is beneficial.

Accordingly, in another aspect the present invention relates to a compound of the general Formula (I) or any optical or geometric isomer or tautomeric form thereof including mixtures of these or a pharmaceutically acceptable salt thereof for use as a pharmaceutical composition.

The invention also relates to pharmaceutical compositions comprising, as an active ingredient, at least one compound of the Formula (I) or any optical or geometric isomer or tautomeric form thereof including mixtures of these or a pharmaceutically acceptable salt thereof together with one or more pharmaceutically acceptable carriers or diluents

In the following synthetic examples, all of the starting materials were obtained from commercial sources unless otherwise indicated. Without further elaboration, it is believed that one skilled in the art can, using the preceding description, utilize the present invention to its fullest extent. These examples are given to illustrate the invention, not to limit its scope.

### EXAMPLES

Materials and Methods

The starting materials used herein are commercially available or can be prepared according to procedures previously reported in the literature. Unless otherwise stated commercial starting materials were used without further purification. All solvents were HPLC grade. Anhydrous solvents were obtained by storing over 4 Å activated molecular sieves. Synthetic methods to prepare the compounds of this invention might employ protective groups to mask a reactive functionality or minimize unwanted side reactions. Such protective groups are described generally in Green (1981).

NMR data were acquired on a Bruker Advance DRX 250. CDCl₃ is deuteriochloroform, DMSO-d₆ is hexadeuteriodimethylsulfoxide, D₂O is deuteriooxide, acetone-d₆ is hexadeuterioacetone and CD₃OD is tetradeuteriomethanol. Abbreviation for NMR data are as follows: s=singlet, d=doublet, t=triplet, q=quartet, h=heptet, m=multiplet. Chemical shifts are reported in ppm, relative to internal solvent peaks (2.50 for DMSO-d₆, 7.26 for CDCl₃, 4.79 for D₂O, 3.31 for CD₃OD). Coupling constants J are reported in Hz. ES-MS spectra were obtained on a Micromass Quattro micro™ instrument in the positive mode unless otherwise noted. Analytical HPLC was performed on a Gilson system (UV/VIS-155 detector at 215 and 254 nm, 402 syringe pump, 819 injection module, valvemate 35, 864 degasser, 233 XL on-line column switching module, and a Zorbax 300SB RP-18 column, 4.6×50 mm with a 322 pump). Eluents A (0.1% TFA in water) and B (1% TFA in acetonitrile) were used in a linear gradient (0% B→100% B in 7 min.). Preparative HPLC was performed on the same Gilson system, using a Zorbax 300SB RP-18, 21.2 mm×25 cm column, with a flow of 15 mL/min.

Abbreviations

AcOH Acetic acid

BPO Benzoylperoxide

DCM Dichloromethane

DMF N,N-Dimethyl formamide

DMSO Dimethyl sulfoxide

m-CPBA m-Chloroperbenzoic acid

NBS N-Bromosuccinimide

THF Tetrahydrofurane

Synthesis of Compounds of the Invention

Illustrative general methods for the synthesis of the compounds of the invention are described below and illustrated in Scheme A-E.

General Method A (Scheme A).

Step 1: Ethyl-5-methylisoxazole-3-carboxylate (6.24 g, 40 mmol) was dissolved in dry CCl₄ (50 mL), and the solution was heated to reflux. A mixture of finely powdered NBS (freshly recrystallized, 7.1 g, 1 mmol) and BPO (freshly recrystallized, 0.5 g, 0.05 mmol) was added in portions over 1 h to the refluxing solution. The mixture was refluxed for 2 h, after which an additional 1.4 g of NBS (0.2 mmol) and 0.5 g of BPO (0.05 mmol) were added in one portion. Refluxing was continued 2 h more. The mixture was cooled down to 0° C., the white flakes were filtered off and the solution concentrated to dryness. Purification of the crude mixture by column chromatography (heptane/EtOAc 97:3→80:20) afforded a colourless oil that slowly crystallized on cooling, as a 85:15 mixture of expected brominated product and unreacted starting material (the two products run very closed on TLC and are difficult to separate completely). The crystals were washed with ether to remove the last traces of impurities. Alternatively, the unreacted starting material may be removed by bulb-tube distillation (100° C. at 0.3 mbar; Micetich et al., 1985). The pure product was obtained in 36% yield (3.4 g, colourless crystals).

Step 2: The product of Step 1 (234 mg, 1 mmol) was dissolved in dry DMF, under argon atmosphere. K₂CO₃ (138 mg, 1.5 mmol) and a thiol (1.1 mmol) were added, and the mixture was stirred at 60° C. for 1-3 h. The solution was cooled to room temperature, water was added and the mixture was extracted 3 times with EtOAc. The combined organic layers were dried over Na₂SO₄, filtered and concentrated to dryness. Purification by flash column chromatography afforded the expected products.

Step 3: The preparation of NH₂OK/NH₂OH solution was performed according to previously reported procedure (Hauser and Renfrow, 1943; Hanessian et al., 2001). In separate vials, under argon, NH₂OH.HCl (28 mg, 4 mmol) in MeOH (2 mL) and KOH (34 mg, 6 mmol) in MeOH (1 mL) were heated to reflux until homogeneous. After cooling below 40° C., the alkali solution was added in one portion to the hydroxylamine solution, and sudden precipitation of potassium chloride was observed. After stirring for 5 min, the ethyl ester prepared at Step 2 (1 mmol) dissolved in MeOH (1 mL) was added in one portion to the suspension. Stirring was continued at room temperature for 1-12 h, until all starting material disappeared on TLC. 1N HCl was added until pH 2, the volume was reduced to 3 mL, and the solution directly purified by preparative HPLC.

Examples 1-18 were prepared by following the General Method A.

General Method B (Scheme B).

Intermediates obtained from Step 2, Scheme A (0.1 mmol) were dissolved in MeOH (1.5 mL) and 1N NaOH (0.5 mL, 0.5 mmol) was added. Stirring was continued at room temperature for 1 h, until no more starting material was visible on TLC. 1N HCl was added until pH 2 and the water phase was extracted with EtOAc. The collected organic layers were washed with NaCl, dried over Na₂SO₄ and concentrated to dryness. In most cases analytically pure products were obtained without need of further purification. Otherwise, recrystallization from heptane/EtOAc or purification by preparative HPLC afforded analytically pure products.

Examples 19-32 were prepared by following the General Method B.

General Method C (Scheme C).

Step 1: Intermediates obtained from Step 2, General Method A (1 mmol), were dissolved in dry DCM, and m-CPBA (690 mg, 2.5 mmol) was added in one portion. Stirring was continued at room temperature for 1-3 h, until total conversion to the di-oxidized product (monitored by TLC and ES-MS). More DCM was added, the organic phase was extracted 3 times with an aq. solution of Na₂CO₃ and once with water, dried over Na₂SO₄, filtered and concentrated to dryness. Purification by flash column chromatography or by recrystallization from heptane/EtOAc afforded the expected products.

Step 2: Same procedure as in Step 3, General Method A.

Examples 33-37 were prepared by following the General Method C.

General Method D (Scheme D).

Step 1: Intermediates obtained from Step 2, General Method A (1 mmol) were dissolved in glacial AcOH (8 mL) and NaBO₃.H₂O (229 mg, 1.5 mmol) was added in one portion. Stirring was continued at room temperature for 1-3 h, monitoring the reaction by ES-MS and TLC. A saturated solution of NaCl was added, extracted 3 times with EtOAc. The collected organic layers were dried over Na₂SO₄, filtered and concentrated to dryness. Purification by flash column chromatography afforded the expected products.

Step 2: Same procedure as in Step 3, General Method A.

Example 38 was prepared by following the General Method D.

General Method E (Scheme E).

Step 1: Intermediates obtained from Step 1, General Method C (0.5 mmol), were dissolved in THF (5 mL), cooled to −30° C. and NaH (0.6 mmol) was added in one portion. After stirring at low temperature for 15 min, an electrophile (1.5 mmol, for example MeI, EtBr, i-PrBr) was slowly added to the solution. After stirring at low temperature for 15-30 minutes, the cooling bath was removed and the solution was allowed to warm to room temperature. Stirring was continued at room temperature for 1-2 h. Water was added, the mixture extracted 3 times with EtOAc, dried over Na₂SO₄, filtered and concentrated to dryness. Purification by flash column chromatography (heptane/EtOAc) or HPLC afforded the expected product.

Step 2: Same procedure as in Step 3, General Method A.

Examples 39-42 were prepared by following the General Method E.

The compounds and processes of the invention are further illustrated by the following non-limiting examples.

### Example 1

### 5-(4-Toluenesulfanylmethyl)-isoxazole-3-carboxylic acid hydroxyamide

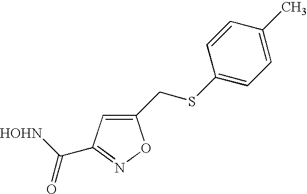

General Method A, Step 2, by using 4-thiocresol (136 mg, 1.1 mmol) as starting material. Yield (after flash column chromatography) 81%. Colourless oil. ¹H NMR (250 MHz, CDCl₃) δ 7.40 (dt, J=8.1 Hz, 2H), 7.24 (broad d, J=8.1 Hz, 2H), 6.57 (s, 1H), 4.55 (q, J=7.2 Hz, 2H), 4.24 (s, 2H), 2.46 (s, 3H), 1.53 (t, J=7.2 Hz, 2H). ¹³C NMR (250 MHz, CDCl₃) δ 171.6, 159.8, 156.4, 138.2, 131.9, 130.0, 129.9, 103.1, 62.1, 30.4, 21.1, 14.1. ES-MS for C₁₄H₁₅NO₃S: 278.0 [M+H]⁺.

General Method A, Step 3. Yield 70%. Off-white crystals. Mp 94-96° C. (decompose). ¹H NMR (500 MHz, CD₃OD) δ 7.26 (d, J=8.5 Hz, 2H), 7.12 (d, J=8.5 Hz, 2H), 6.38, (s, 1H), 4.21 (s, 2H), 2.29 (s, 3H). ¹³C NMR (500 MHz, CD₃OD) δ 173.0, 158.9, 158.3, 139.1, 132.8, 131.5, 130.9, 102.7, 30.4, 21.0. ES-MS for C₁₂H₁₂N₂O₃S: 265.0 [M+H]⁺.

### Example 2

### 5-(4-Chloro-phenylsulfanylmethyl)-isoxazole-3-carboxylic acid hydroxyamide

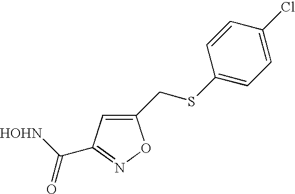

General Method A, Step 2, by using 4-chlorothiophenol (144 mg, 1.1 mmol) as starting material. Yield (after flash column chromatography) 89%. White crystals. ¹H NMR (250 MHz, CDCl₃) δ 7.30 (s, 4H), 6.49 (s, 1H), 4.45 (q, J=7.1 Hz, 2H), 4.17 (d, J=0.7 Hz, 2H), 1.43 (t, J=7.1 Hz, 2H). ¹³C NMR (250 MHz, CDCl₃) δ 170.8, 159.4, 156.3, 133.8, 132.3, 131.9, 129.2, 103.1, 62.0, 29.7, 13.9. ES-MS for C₁₃H₁₂ClNO₃S: 298.0 [M+H]⁺.

General Method A, Step 3. Yield 65%. Off-white crystals. ¹H NMR (250 MHz, CD₃OD) δ 7.37 (m, 4H), 6.48 (s, 1H), 4.33 (s, 2H). ¹³C NMR (250 MHz, CD₃OD) δ 172, 158.1, 134.3, 133.9, 133.2, 129.9, 102.6, 29.5 (one signal missing). ES-MS for C₁₁H₉ClN₂O₃S: 285.0 [M+H]⁺.

### Example 3

### 5-(3-Chloro-phenylsulfanylmethyl)-isoxazole-3-carboxylic acid hydroxyamide

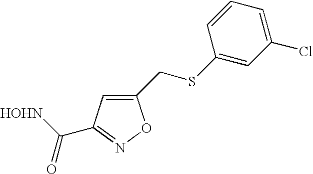

General Method A, Step 2, by using 3-chlorothiophenol (144 mg, 1.1 mmol) as starting material. Yield (after flash column chromatography) 74%. Colourless oil. ¹H NMR (250 MHz, CDCl₃) δ 7.55 (m, 1H), 7.42 (m, 3H), 6.70 (s, 1H), 4.62 (q, J=7.1 Hz, 2H), 4.39 (s, 2H), 1.60 (t, J=7.1 Hz, 3H). ¹³C NMR (250 MHz, CDCl₃) δ 170.5, 159.3, 156.2, 135.6, 134.5, 130.0, 129.8, 128.1, 127.4, 103.1, 61.9, 28.9, 13.8. ES-MS for C₁₃H₁₂ClNO₃S: 298.0 [M+H]⁺.

General Method A, Step 3. Yield 75%. Pale yellow crystals. Mp 105-107° C. (decompose). ¹H NMR (250 MHz, CD₃OD) δ 7.40 (m, 1H), 7.28 (m, 3H), 6.50 (s, 1H), 4.35 (s, 2H). ¹³C NMR (250 MHz, CD₃OD) δ 172.8, 159.2, 158.9, 138.2, 136.2, 131.9, 131.2, 130.0, 128.8, 103.4, 29.7. ES-MS for C₁₁H₉ClN₂O₃S: 285.0 [M+H]⁺.

### Example 4

### 5-(2-Chloro-phenylsulfanylmethyl)-isoxazole-3-carboxylic acid hydroxyamide

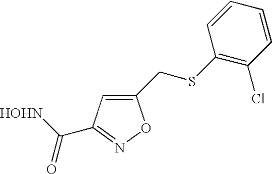

General Method A, Step 2, by using 2-chlorothiophenol (144 mg, 1.1 mmol) as starting material. Yield (after flash column chromatography) 70%. White crystals. Mp 57° C. ¹H NMR (250 MHz, CDCl₃) δ 7.33 (m, 1H), 7.21 (m, 1H), 7.11 (m, 2H), 6.41 (d, J=0.7 Hz, 1H), 4.32 (q, J=7.1 Hz, 2H), 4.15 (d, J=0.7 Hz, 2H), 1.31 (t, J=7.1 Hz, 3H). ¹³C NMR (250 MHz, CDCl₃) δ 170.5, 159.4, 156.3, 135.1, 132.4, 131.1, 129.8, 128.4, 127.2, 103.2, 61.9, 27.8, 13.9. ES-MS for C₁₃H₁₂ClNO₃S: 298.0 [M+H]⁺.

General Method A, Step 3. Yield 86%. White crystals. Mp 141-143° C. (decompose). ¹H NMR (250 MHz, CD₃OD) δ 7.42 (m, 2H), 7.21 (m, 2H), 6.50 (s, 1H), 4.38 (s, 2H). ¹³C NMR (250 MHz, CD₃OD) δ 172.1, 158.7, 158.4, 135.8, 134.4, 132.0, 130.9, 129.4, 128.6, 103.0, 28.2. ES-MS for C₁₁H₉ClN₂O₃S: 285.1 [M+H]⁺.

### Example 5

### 5-(3,4-Dichloro-phenylsulfanylmethyl)-isoxazole-3-carboxylic acid hydroxyamide

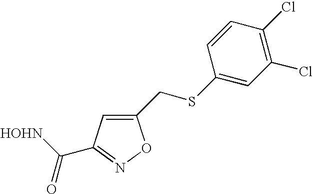

General Method A, Step 2, by using 3,4-dichlorothiophenol (196 mg, 1.1 mmol) as starting material. Yield (after flash column chromatography) 81%. Colourless oil. ¹H NMR (250 MHz, CDCl₃) δ 7.43 (d, J=2.1 Hz, 1H), 7.34 (d, J=8.4 Hz, 1H), 7.15 (dd, J=8.4, 2.1 Hz, 1H), 6.5 (s, 1H), 4.40 (q, J=7.1 Hz, 2H), 4.18 (s, 2H), 1.39 (t, J=7.1 Hz, 3H). ¹³C NMR (250 MHz, CDCl₃) δ 170.1, 159.2, 156.1, 133.7, 132.6, 131.6, 131.4, 130.5, 129.4, 103.0, 61.8, 29.0, 13.7. ES-MS for C₁₃H₁₁Cl₂NO₃S: 332.0 [M+H]⁺.

General Method A, Step 3. Yield 79%. Off-white crystals. Mp 99-102° C. (decompose). ¹H NMR (250 MHz, CD₃OD) δ 7.54 (d, J=2.1 Hz, 1H), 7.44 (d, J=8.4 Hz, 1H), 7.29 (dd, J=8.4, 2.1 Hz, 1H), 6.53 (s, 1H), 4.37 (s, 2H). ¹³C NMR (250 MHz, CD₃OD) δ 172.1, 158.7, 158.4, 136.2, 133.8, 132.8, 132.3, 132.0, 131.0, 103.1, 29.3. ES-MS for C₁₁H₈Cl₂N₂O₃S: 319.0 [M+H]⁺.

### Example 6

### 5-(2-Bromo-phenylsulfanylmethyl)-isoxazole-3-carboxylic acid hydroxyamide

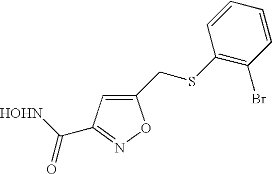

General Method A, Step 2, by using 2-bromothiophenol (207 mg, 1.1 mmol) as starting material. Yield (after flash column chromatography) 90%. White crystals. Mp 58-59° C. ¹H NMR (250 MHz, CDCl₃) δ 7.13 (m, 1H), 7.30 (m, 2H), 7.15 (m, 1H), 6.55 (s, 1H), 4.45 (q, J=7.1 Hz, 2H), 4.27 (d, J=0.7 Hz, 2H), 1.41 (t, J=7.1 Hz, 3H). ¹³C NMR (250 MHz, CDCl₃) δ 170.4, 159.5, 156.3, 134.5, 133.2, 130.7, 128.4, 127.9, 125.3, 103.3, 62.0, 28.2, 13.9. ES-MS for C₁₃H₁₂BrNO₃S: 341.9 [M+H]⁺.

General Method A, Step 3. Yield 89%. White crystals. Mp 135-136° C. (decompose). ¹H NMR (250 MHz, CD₃OD) δ 7.66 (dd, J=8.0, 1.3 Hz, 1H), 7.47 (dd, J=7.8, 1.3 Hz, 1H), 7.36 (m, 1H), 7.19 (m, 1H), 6.59 (s, 1H), 4.45 (2, 2H). ¹³C NMR (250 MHz, CD₃OD) δ 172.0, 158.7, 158.4, 136.5, 134.3, 131.5, 129.3, 129.2, 125.7, 103.1, 28.6. ES-MS for C₁₁H₉BrN₂O₃S: 328.9 [M+H]⁺.

### Example 7

### 5-(3-Bromo-phenylsulfanylmethyl)-isoxazole-3-carboxylic acid hydroxyamide

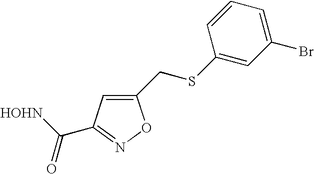

General Method A, Step 2, by using 3-bromothiophenol (207 mg, 1.1 mmol) as starting material. Yield (after flash column chromatography) 82%. Colourless oil. ¹H NMR (250 MHz, CDCl₃) δ 7.53 (dt, J=1.8, 0.3 Hz, 1H), 7.40 (ddd, J=7.8, 1.8, 1.3 Hz, 1H), 7.27 (ddd, J=7.8, 1.8, 1.3 Hz, 1H), 7.17 (dt, J=7.8, 0.3 Hz, 1H), 6.52 (t, J=0.7 Hz, 1H), 4.47 (q, J=7.1 Hz, 2H), 4.21 (d, J=0.7 Hz, 2H), 1.42 (t, J=7.1 Hz, 3H). ¹³C NMR (250 MHz, CDCl₃) δ 170.5, 159.4, 156.3, 135.8, 132.8, 130.4, 130.3, 128.8, 122.7, 103.2, 62.0, 29.1, 13.9. ES-MS for C₁₃H₁₂BrNO₃S: 341.9 [M+H]⁺.

General Method A, Step 3. Yield 86%. White crystals. Mp 104-105° C. ¹H NMR (250 MHz, CD₃OD) δ 7.55 (t, J=1.8 Hz, 1H), 7.40 (ddd, J=7.8, 1.8, 1.1 Hz, 1H), 7.35 (ddd, J=7.8, 1.8, 1.1 Hz, 1H), 7.22 (t, J=7.8 Hz, 1H), 6.50 (s, 1H), 4.35 (s, 2H). ¹³C NMR (250 MHz, CD₃OD) δ 172.3, 159.0, 158.4, 138.0, 133.7, 131.7, 131.3, 130.0, 123.7, 103.0, 29.3. ES-MS for C₁₁H₉BrN₂O₃S: 328.9 [M+H]⁺.

### Example 8

### 5-(2-Isopropyl-phenylsulfanylmethyl)-isoxazole-3-carboxylic acid hydroxyamide

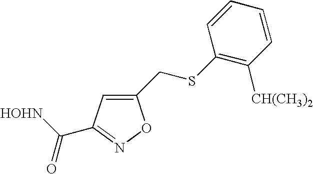

General Method A, Step 2, by using 2-isopropyl-thiophenol (152 mg, 1.1 mmol) as starting material. Yield (after flash column chromatography) 85%. Colourless oil. ¹H NMR (250 MHz, CDCl₃) δ 7.43 (m, 3H), 7.28 (m, 1H), 6.54 (s, 1H), 4.55 (q, J=7.1 Hz, 2H), 4.27 (s, 2H), 3.60 (h, J=6.9 Hz, 1H), 1.54 (t, J=7.1 Hz, 3H), 1.34 (d, J=6.9 Hz, 6H). ¹³C NMR (250 MHz, CDCl₃) δ 171.0, 159.6, 156.2, 149.8, 131.6, 131.5, 128.1, 126.4, 125.8, 103.0, 61.9, 30.2, 29.7, 23.3, 13.9. ES-MS for C₁₆H₁₉NO₃S: 306.0 [M+H]⁺.

General Method A, Step 3. Yield 86%. White crystals. Mp 121-123° C. (decompose). ¹H NMR (250 MHz, CD₃OD) δ 7.42 (m, 1H), 7.27 (m, 2H), 7.14 (m, 1H), 6.34 (s, 1H), 3.44 (h, J=6.9 Hz, 1H), 1.15 (d, J=6.9 Hz, 6H). ¹³C NMR (250 MHz, CD₃OD) δ 173.0, 159.2, 158.8, 151.9, 133.9, 133.5, 129.8, 128.0, 127.3, 103.2, 31.9, 31.0, 24.3. ES-MS for C₁₄H₁₆N₂O₃S: 292.9 [M+H]⁺.

### Example 9

### 5-(2,4,6-Trimethyl-phenylsulfanylmethyl)-isoxazole-3-carboxylic acid hydroxyamide

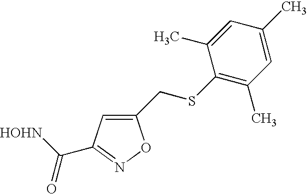

General Method A, Step 2, by using 2,4,6-trimethylbenzen-1-thiol (152 mg, 1.1 mmol) as starting material. Yield (after flash column chromatography) 45%. Colourless oil. ¹H NMR (250 MHz, CDCl₃) δ 6.82 (broad s, 2H), 6.15 (broad t, J=0.5 Hz, 2H), 4.32 (q, J=7.2 Hz, 2H), 3.79 (d, J=0.5 Hz, 2H), 2.27 (s, 6H), 2.16 (s, 3H), 1.31 (t, J=7.1 Hz, 3H). ¹³C NMR (250 MHz, CDCl₃) δ 171.8, 160.2, 156.7, 143.5, 139.6, 129.6, 128.1, 103.2, 62.5, 29.9, 21.9, 21.4, 14.4. ES-MS for C₁₆H₁₉NO₃S: 305.9 [M+H]⁺.

General Method A, Step 3. Yield 84%. White crystals. Mp 133-135° C. (decompose). ¹H NMR (250 MHz, CD₃OD) δ 6.90 (s, 2H), 6.16 (s, 1H), 3.94 (s, 2H), 2.32 (s, 6H), 2.22 (s, 3H). ¹³C NMR (250 MHz, CD₃OD) δ 172.7, 158.8, 158.4, 144.5, 140.4, 130.1, 128.9, 102.6, 29.9, 21.7, 21.1. ES-MS for C₁₄H₁₆N₂O₃S: 293.0 [M+H]⁺.

### Example 10

### 5-(3-Methoxy-phenylsulfanylmethyl)-isoxazole-3-carboxylic acid hydroxyamide

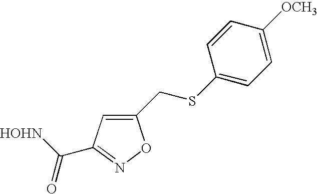

General Method A, Step 2, by using 4-methoxythiophenol (140 mg, 1.1 mmol) as starting material. Yield (after flash column chromatography) 63%. Colourless oil. ¹H NMR (250 MHz, CDCl₃) δ 7.30 (m, 2H), 6.81 (m, 2H), 6.38 (broad t, J=0.6 Hz, 1H), 4.40 (q, J=7.1 Hz, 2H), 4.03 (d, J=0.6 Hz, 2H), 3.78 (s, 3H), 1.39 (t, J=7.1 Hz, 3H). ¹³C NMR (250 MHz, CDCl₃) δ 171.4, 159.8, 159.6, 156.1, 134.6, 123.5, 114.6, 102.8, 61.8, 55.0, 31.2, 13.8. ES-MS for C₁₄H₁₅NO₄S: 293.9 [M+H]⁺.

General Method A, Step 3. Yield 75%. Pale yellow crystals. ¹H NMR (250 MHz, CD₃OD) δ 7.30 (m, 2H), 6.85 (m, 2H), 6.34 (broad s, 1H), 4.12 (d, J=0.4 Hz, 2H), 3.76 (s, 3H). ¹³C NMR (250 MHz, CD₃OD) δ 173.0, 161.5, 158.9, 158.3, 135.9, 125.2, 115.8, 102.7, 55.8, 31.7. ES-MS for C₁₂H₁₂N₂O₄S: 280.9 [M+H]⁺.

### Example 11

### 5-(3-Methoxy-phenylsulfanylmethyl)-isoxazole-3-carboxylic acid hydroxyamide

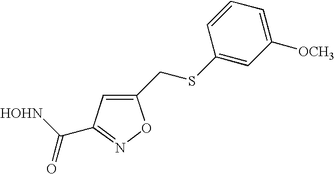

General Method A, Step 2, by using 3-methoxythiophenol (140 mg, 1.1 mmol) as starting material. Yield (after flash column chromatography) 78%. Colourless oil. ¹H NMR (250 MHz, CDCl₃) δ 7.21 (m, 1H), 6.91 (ddd, J=7.6, 1.7, 0.9 Hz, 1H), 6.87 (m, 1H), 6.79 (ddd, J=8.3, 2.5, 0.9 Hz, 1H), 6.49 (t, J=0.7 Hz, 1H), 4.40 (q, J=7.1 Hz, 2H), 4.17 (d, J=0.7 Hz, 2H), 3.77 (s, 3H), 1.39 (t, J=7.1 Hz, 3H). ¹³C NMR (250 MHz, CDCl₃) δ 171.2, 159.8, 159.6, 156.3, 134.8, 129.9, 122.5, 115.8, 113.2, 103.1, 62.0, 55.1, 29.2, 13.9. ES-MS for C₁₄H₁₅NO₄S: 294.0 [M+H]⁺.

General Method A, Step 3. Yield 96%. White crystals. Mp 116° C. ¹H NMR (250 MHz, CD₃OD) δ 7.21 (m, 1H), 6.95 (ddd, J=7.7, 1.6, 0.9 Hz, 1H), 6.91 (m, 1H), 6.81 (ddd, J=8.2, 2.5, 0.9 Hz, 1H), 6.46 (s, 1H), 4.29 (s, 2H), 3.75(s, 3H). ¹³C NMR (250 MHz, CD₃OD) δ 173.3, 161.9, 159.2, 158.8, 137.0, 131.4, 124.1, 117.3, 114.7, 103.3, 56.1, 30.0. ES-MS for C₁₂H₁₂N₂O₄S: 281.1 [M+H]⁺.

### Example 12

### 5-(4-Fluoro-phenylsulfanylmethyl)-isoxazole-3-carboxylic acid hydroxyamide

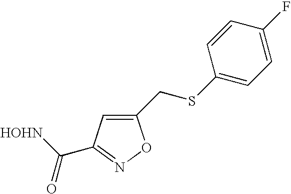

General Method A, Step 2, by using 4-fluorothiophenol (128 mg, 1.1 mmol) as starting material. Yield (after flash column chromatography) 70%. Colourless oil. ¹H NMR (250 MHz, CDCl₃) δ 7.33 (m, 2H), 6.97(m, 2H), 6.39 (s, 1H), 4.39 (q, J=7.1 Hz, 2H), 4.08 (s, 2H), 1.37 (t, J=7.1 Hz, 3H). ¹³C NMR (250 MHz, CDCl₃) δ 171.0, 162.6 (d, J=249 Hz), 159.6, 156.3, 134.4 (d, J=8 Hz), 128.3 116.5 (d, J=22 Hz), 103.0, 62.1, 30.7, 13.9. ES-MS for C₁₃H₁₂FNO₃S: 282.0 [M+H]⁺.

General Method A, Step 3. Yield 77%. Pale orange crystals. Mp 98-99° C. (decompose). ¹H NMR (250 MHz, CD₃OD) δ 7.41 (m, 2H), 7.05 (m, 2H), 6.41 (s, 1H), 4.24 (s, 2H). ¹³C NMR (250 MHz, CD₃OD) δ 172.7, 163.8 (d, J=247 Hz), 158.8, 158.4, 135.5 (d, J=8 Hz), 130.5 (d, J=3 Hz), 117.4 (d, J=22 Hz), 102.9, 30.8. ES-MS for C₁₁H₉FN₂O₃S: 269.0 [M+H]⁺.

### Example 13

### 5-(4-Trifluoromethyl-phenylsulfanylmethyl)-isoxazole-3-carboxylic acid hydroxyamide

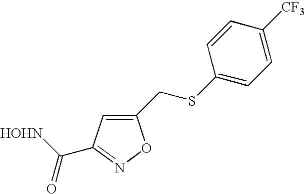

General Method A, Step 2, by using 4-(trifluoromethyl)benzenthiol (178 mg, 1.1 mmol) as starting material. Yield (after flash column chromatography) 78%. Pale yellow crystals. Mp 50-51° C. ¹H NMR (250 MHz, CDCl₃) δ 7.55 (d, J=8.8 Hz, 2H), 7.40 (d, J=8.8 Hz, 2H), 6.54 (t, J=0.7 Hz, 1H), 4.42 (q, J=7.1 Hz, 2H), 4.25 (d, J=0.7 Hz, 2H), 1.39 (t, J=7.1 Hz, 2H). ES-MS for C₁₄H₁₂F₃NO₃S: 332.0 [M+H]⁺.

General Method A, Step 3. Yield 80%. White crystals. Mp 129° C. ¹H NMR (250 MHz, CD₃OD) δ 7.78 (m, 4H), 6.79 (s, 1H), 4.68 (s, 2H). ES-MS for C₁₂H₉F₃N₂O₃S: 319.0 [M+H]⁺.

### Example 14

### 5-(2-Trifluoromethyl-phenylsulfanylmethyl)-isoxazole-3-carboxylic acid hydroxyamide

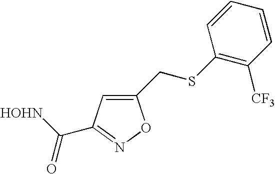

General Method A, Step 2, by using 2-(trifluoromethyl)benzenthiol (178 mg, 1.1 mmol) as starting material. Yield (after flash column chromatography) 78%. Pale yellow oil. ¹H NMR (250 MHz, CDCl₃) δ 7.60 (m, 1H), 7.36 (m, 3H), 6.39 (s, 1H), 4.33 (q, J=7.1 Hz, 2H), 4.13 (s, 2H), 1.31 (t, J=7.1 Hz, 3H). ¹³C NMR (250 MHz, CDCl₃) δ 170.4, 159.6, 156.4, 136.8, 133.8, 132.2, 129.3, 127.9, 127.0 (q, J=5 Hz), 123.3 (q, J=273 Hz), 103.4, 62.1, 29.9, 13.9. ES-MS for C₁₄H₁₂F₃NO₃S: 332.0 [M+H]⁺.

General Method A, Step 3. Yield 56%. White crystals. Mp 140-144° C. (decompose). ¹H NMR (250 MHz, CD₃OD) δ 7.70 (broad d, J=7.8 Hz, 1H), 7.65 (broad d, J=7.8 Hz, 1H), 7.57 (broad t, J=7.5 Hz, 1H), 7.43 (broad t, J=7.5 Hz, 1H), 6.45 (s, 1H), 4.40 (s, 1H). ¹³C NMR (250 MHz, CD₃OD) δ 172.3, 159.1, 158.8, 136.8, 134.9, 134.1, 129.1, 128.3 (q, J=5 Hz), 125.4 (q, J=273 Hz), 103.5, 30.4 (one signal missing). ES-MS for C₁₂H₁₉F₃N₂O₃S: 318.9 [M+H]⁺.

### Example 15

### 5-(2-Trifluoromethoxy-phenylsulfanylmethyl)-isoxazole-3-carboxylic acid hydroxyamide

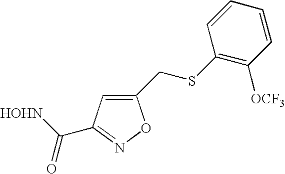

General Method A, Step 2, by using 2-(trifluoromethoxy)benzenthiol (194 mg, 1.1 mmol) as starting material. Yield (after flash column chromatography) 84%. Pale yellow oil. ¹H NMR (250 MHz, CDCl₃) δ 7.30-7.14 (m, 4H), 6.43 (t, J=0.7 Hz, 1H), 4.36 (q, J=7.1 Hz, 2H), 4.15 (d, J=0.7 Hz, 2H), 1.34 (t, J=7.1 Hz, 3H). ¹³C NMR (250 MHz, CDCl₃) δ 170.5, 159.5, 156.3, 148.1, 132.2, 128.9, 127.2, 126.9, 120.9, 120.2 (d, J=259 Hz), 103.1, 61.9, 27.9, 13.8. ES-MS for C₁₄H₁₂F₃NO₄S: 347.9 [M+H]⁺.

General Method A, Step 3. Yield 73%. White crystals. Mp 108-109° C. (decompose). ¹H NMR (250 MHz, CD₃OD) δ 7.52 (m, 1H), 7.34 (m, 3H), 6.48 (s, 1H), 4.36 (s, 2H). ¹³C NMR (500 MHz, CD₃OD) δ 172.0, 158.4, 149.0, 132.8, 129.7, 129.1, 128.7, 122.1, 102.7, 27.9 (2 signals missing). ES-MS for C₁₂H₉F₃N₂O₄S: 335.0 [M+H]⁺.

### Example 16

### 5-(Benzothiophen-2-ylmethylsulfanylmethyl)-isoxazole-3-carboxylic acid hydroxyamide

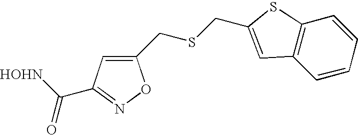

General Method A, Step 2, by using benzothiophen-3-ylmethanthiol (180 mg, 1.1 mmol) as starting material. Yield (after flash column chromatography) 65%. Pale pink crystals. Mp 78-79° C. ¹H NMR (250 MHz, CDCl₃) δ 7.80 (m, 2H), 7.34 (m, 3H), 6.45 (s, 1H), 4.39 (q, J=7.1 Hz), 3.96 (s, 2H), 3.61 (s, 2H), 1.38 (t, J=7.1 Hz, 3H). ¹³C NMR (250 MHz, CDCl₃) δ 171.9, 159.5, 156.3, 140.4, 137.3, 130.3, 124.8, 124.4, 123.9, 122.7, 121.9, 102.5, 61.9, 29.3, 25.2, 13.9. ES-MS for C₁₆H₁₅NO₃S₂: 334.0 [M+H]⁺.

General Method A, Step 3. Yield 75%. White crystals. Mp 143-145° C. (decompose). ¹³C NMR (250 MHz, CD₃OD) δ 173,4; 158,9; 158,5; 142,0; 139,1; 132,4; 125,9; 125,6; 125,0; 123,8; 123,3; 102,5; 30,3; 26,3. ES-MS for C₁₄H₁₂N₂O₃S₂: 321.0 [M+H]⁺.

### Example 17

### 5-(3-Trifluoromethyl-benzylsulfanylmethyl)-isoxazole-3-carboxylic acid hydroxyamide

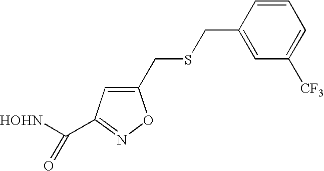

General Method A, Step 2, by using [3-(trifluoromethyl)phenyl] (192 mg, 1.1 mmol) as starting material. Yield (after flash column chromatography) 84%. Pale pink oil. ¹H NMR (250 MHz, CDCl₃) δ 7.49 (m, 4H), 6.51 (s, 1H), 4.44 (q, J=7.1 Hz, 2H), 3.81 (s, 2H), 3.69 (s, 2H), 1.41 (t, J=7.1 Hz, 3H). ¹³C NMR (250 MHz, CDCl₃) δ 172.1, 160.0, 156.9, 138.4, 132.7, 131.5 (q, J=32 Hz), 129.5, 126.0 (q, J=4 Hz), 124.6 (q, J=4 Hz), 124.2 (q, J=272 Hz), 103.3, 62.5, 36.2, 25.8, 14.4. ES-MS for C₁₅H₁₄F₃NO₃S: 345.8 [M+H]⁺.

General Method A, Step 3. Yield 85%. Pink crystals. Mp 109-110° C. ¹H NMR (250 MHz, CD₃OD) δ 7.62 (s, 1H), 7.53 (m, 3H), 6.53 (s, 1H), 3.87 (s, 2H), 3.79 (s, 2H). ¹³C NMR (250 MHz, CD₃OD) δ 173.2, 158.9, 158.5, 140.4, 133.8, 131.9 (q, J=32 Hz), 130.4, 126.7 (q, J=4 Hz), 125.5 (q, J=272 Hz), 125.0 (q, J=4 Hz), 102.7, 36.7, 26.3. ES-MS for C₁₃H₁₁F₃N₂O₃S: 332.9 [M+H]⁺.

### Example 18

### 5-(4-Thiophen-2-yl-pyrimidin-2-ylsulfanylmethyl)-isoxazole-3-carboxylic acid hydroxyamide

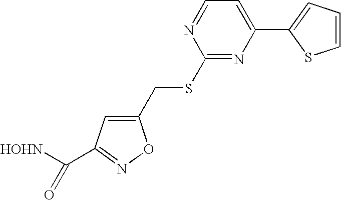

General Method A, Step 2, by using 4-(2-thienyl)pyrimidine-2-thiol (194 mg, 1.1 mmol) as starting material. Yield (after flash column chromatography) 82%. Pale yellow crystals. Mp 76-77° C. ¹H NMR (250 MHz, CDCl₃) δ 8.40 (d, J=5.3 Hz, 1H), 7.68 (dd, J=3.8, 1.1 Hz, 1H), 7.49 (dd, J=5.0, 1.1 Hz, 1H), 7.20 (d, J=5.3 Hz, 1H), 7.08 (dd, J=5.0, 3.8 Hz, 1H), 6.68 (t, J=0.7 Hz, 1H), 4.49 (d, J=0.7 Hz, 2H), 4.33 (q, J=7.1 Hz, 2H), 1.32 (t, J=7.1 Hz, 3H). ¹³C NMR (250 MHz, CDCl₃) δ 172.2, 170.0, 160.2, 159.6, 158.1, 156.8, 141.8, 131.3, 128.9, 128.6, 111.4, 103.5, 62.4, 25.9, 14.4. ES-MS for C₁₅H₁₃N₃O₃S₂: 348.0 [M+H)⁺.

General Method A, Step 3. Yield 78%. White crystals. Mp 165-168° C. (decompose). ¹H NMR (250 MHz, CD₃OD) δ 8.50 (d, J=5.4 Hz, 1H), 7.93 (dd, J=3.8, 1.1 Hz, 1H), 7.70 (dd, J=5.0, 1.1 Hz, 1H), 7.53 (d, J=5.4 Hz, 1H), 7.20 (dd, J=5.0, 3.8 Hz, 1H), 6.70 (broad s), 4.62 (broad s, 2H). ES-MS for C₁₃H₁₀N₄O₃S₂: 335.0 [M+H]⁺.

### Example 19

### 5-(4-Toluenesulfanylmethyl)-isoxazole-3-carboxylic acid

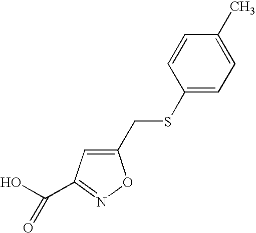

General Method B. Yield 84%. White crystals. ¹H NMR (250 MHz, CDCl₃) δ 7.27 (m, 2H), 7.11 (broad d, J=7.9 Hz, 2H), 6.47 (s, 1H), 4.12 (s, 2H), 2.33 (s, 3H). ES-MS for C₁₂H₁₁NO₃S: 249.9 [M+H]⁺.

### Example 20

### 5-(4-Chloro-phenylsulfanylmethyl)-isoxazole-3-carboxylic acid

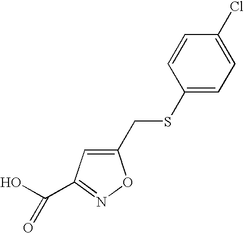

General Method B. Yield 75%. White crystals. ¹H NMR (250 MHz, CDCl₃) δ 7.29 (s, 4H), 6.51 (s, 1H), 4.16 (s, 2H). ES-MS for C₁₁H₈ClNO₃S: 269.9 [M+H]⁺.

### Example 21

### 5-(3-Chloro-phenylsulfanylmethyl)-isoxazole-3-carboxylic acid

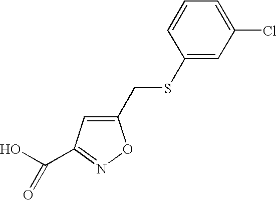

General Method B. Yield 98%. White crystals. ¹H NMR (250 MHz, CDCl₃) δ 7.35 (m, 1H), 7.23 (m, 3H), 6.54 (t, J=0.7 Hz, 1H), 4.20 (d, J=0.7 Hz, 2H). ES-MS for C₁₁H₈ClNO₃S: 269.9 (M+H]⁺.

### Example 22

### 5-(2-isopropyl-phenylsulfanylmethyl)-isoxazole-3-carboxylic acid

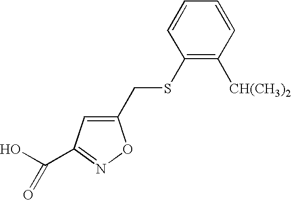

General Method B. Yield 98%. White crystals. ¹H NMR (250 MHz, CDCl₃) δ 7.21 (m, 3H), 7.06 (m, 1H), 6.35 (s, 1H), 4.07 (s, 2H), 3.38 (h, J=6.9 Hz, 1H), 1.12 (d, J=6.9 Hz, 6H). ES-MS for C₁₄H₁₅NO₃S: 277.9 [M+H]⁺.

### Example 23

### 5-(2,4,6-Trimethyl-phenylsulfanylmethyl)-isoxazole-3-carboxylic acid

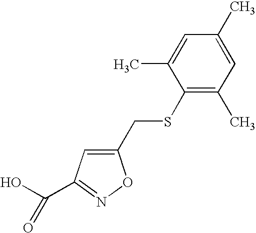

General Method B. Yield 96%. White foam. ¹H NMR (250 MHz, CD₃OD) δ 6.92 (s, 2H), 6.27 (d, 1H), 3.90 (s, 2H), 2.37 (s, 6H), 2.26 (s, 3H). ¹³C NMR (250 MHz, CD₃OD) δ 172.1, 163.6, 155.6, 143.1, 139.3, 129.2, 127.5, 102.9, 29.4, 21.5, 21.0. ES-MS for C₁₄H₁₅NO₃S: 278.0 [M+H]⁺.

### Example 24

### 5-(4-Methoxy-phenylsulfanylmethyl)-isoxazole-3-carboxylic acid

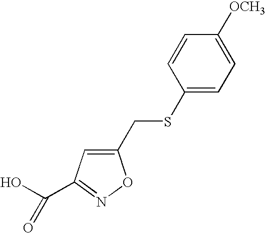

General Method B. Yield 87%. White crystals. ¹H NMR (250 MHz, CD₃OD) δ 7.34 (d, J=8.8 Hz, 2H), 6.89 (d, J=8.8 Hz, 2H), 6.39 (s, 1H), 4.93 (s, 2H), 3.87 (s, 3H). ¹³C NMR (250 MHz, CD₃OD) δ 173.9, 162.8, 162.0, 158.6, 136.4, 125.6, 116.2, 104.4, 56.2, 32.1. ES-MS for C₁₂H₁₁NO₄S: 266.0 [M+H]⁺.

### Example 25

### 5-(3-Methoxy-phenylsulfanylmethyl)-isoxazole-3-carboxylic acid

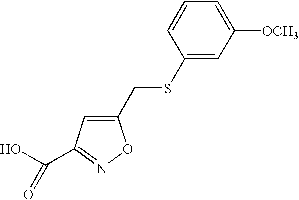

General Method B. Yield 100%. White crystals. ¹H NMR (250 MHz, CD₃OD) δ 7.1 (t, J=8.1 Hz, 1H), 6.86 (dd, J=1.7, 0.9 Hz, 1H), 6.81 (m, 1H), 6.71 (ddd, J=8.1, 2.5, 0.9 Hz, 1H), 4.19 (s, 2H), 3.16 (s, 3H). ¹³C NMR (250 MHz, CD₃OD) δ 162.9, 161.9, 158.8, 136.9, 131.5, 124.2, 117.4, 114.7, 104.6, 56.2, 30.1 (one quaternary signal missing). ES-MS for C₁₂H₁₁NO₄S: 266.0 [M+H]⁺.

### Example 26

### 5-(4-Fluoro-phenylsulfanylmethyl)-isoxazole-3-carboxylic acid

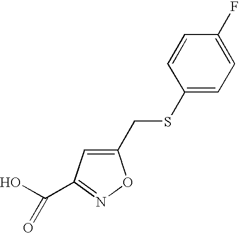

General Method B. Yield 100%. White crystals. ¹H NMR (250 MHz, CD₃OD) δ 7.40 (m, 2H), 7.08 (m, 2H), 6.49 (s, 1H), 4.27 (s, 2H). ¹³C NMR (250 MHz, CD₃OD) δ 173.1, 163.8 (d, J=247 Hz), 162.2, 158.1, 135.4 (d, J=8 Hz), 130.2, 117.3 (d, J=22 Hz), 104.1, 30.9. ES-MS for C₁₁H₁₈FNO₃S: 253.9 [M+H]⁺.

### Example 27

### 5-(4-Trifluoromethyl-phenylsulfanylmethyl)-isoxazole-3-carboxylic acid

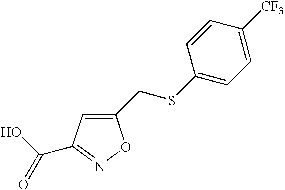

General Method B. Yield 98%. White crystals. ¹H NMR (250 MHz, CDCl₃) δ 7.56 (d, J=8.2 Hz, 2H), 7.41 (d, J=8.2 Hz, 2H), 7.26, 6.59 (t, J=0.7 Hz, 1H), 4.27 (d, J=0.7 Hz, 2H). ES-MS for C₁₂H₈F₃NO₃S: 303.9 [M+H]⁺.

### Example 28

### 5-(2-Trifluoromethyl-phenylsulfanylmethyl)-isoxazole-3-carboxylic acid

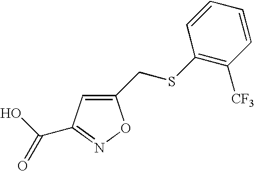

General Method B. Yield after recrystallization from heptane/EtOAc 61%. White crystals. ¹H NMR (250 MHz, CDCl₃) δ 9.24 (broad s, 1H), 7.72 (m, 1H), 7.48 (m, 1H), 7.41 (m, 2H), 6.51 (s, 1H), 4.23 (s, 2H). ¹³C NMR (250 MHz, CDCl₃) δ 171.3, 163.1, 155.6, 134.7, 134.2, 133.4, 132.3, 128.2, 127.3, 103.6, 30.1 (signal of CF₃ missing). ES-MS for C₁₂H₈F₃NO₃S: 303.9 [M+H]⁺.

### Example 29

### 5-(2-Trifluoromethoxy-phenylsulfanylmethyl)-isoxazole-3-carboxylic acid

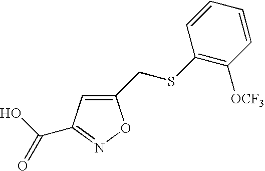

General Method B. Yield after recrystallization 81%. White crystals. ¹H NMR (250 MHz, CDCl₃) δ 7.38-7.20 (m, 4H), 6.49 (s, 1H), 4.23 (s, 2H). ¹³C NMR (250 MHz, CD₃OD) δ 173.0, 162.7, 158.7, 154.2, 133.8, 130.5, 129.3, 128.0, 122.8, 104.6, 28.8 (signal of OCF3 and COCF3 missing). ES-MS for C₁₂H₁₈F₃NO₄S: 319.8 [M+H]⁺.

### Example 30

### 5-(Benzothiophen-2-ylmethylsulfanylmethyl)-isoxazole-3-carboxylic acid

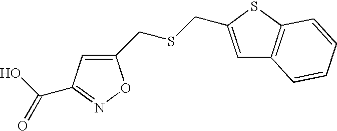

General Method B. Yield 100%. White crystals. ¹H NMR (250 MHz, CD₃OD) δ 7.85 (m, 2H), 7.41 (broad s, 1H), 7.34 (m, 2H), 6.46 (t, J=0.7 Hz, 1H), 4.05 (d, J=0.8 Hz, 2H), 3.77 (d, J=0.7 Hz, 2H). ¹³C NMR (250 MHz, CD₃OD) δ 174.0, 162.3, 158.2, 142.0, 139.1, 132.5, 126.0, 125.6, 125.0, 123.8, 123.3, 103.7, 30.5, 26.4. ES-MS for C₁₄H₁₁NO₃S₂: 305.9 [M+H]⁺.

### Example 31

### 5-(3-Trifluoromethyl-benzylsulfanylmethyl)-isoxazole-3-carboxylic acid

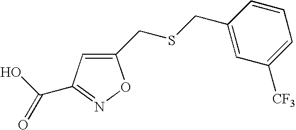

General Method B. Yield 100%. White crystals. ¹H NMR (250 MHz, CDCl₃) δ 9.93 (broad s, 1H), 7.59 (broad s, 1H), 7.49 (m, 3H), 6.58 (s, 1H), 3.82 (s, 2H), 3.71 (s, 2H). ¹³C NMR (250 MHz, CDCl₃) δ 172.5, 163.5, 155.8, 137.8, 132.3, 131.1 (q, J=32 Hz), 129.2, 125.6 (q, J=4 Hz), 124.4 (q, J=4 Hz), 103.1, 36.0, 25.5 (signal of CF₃ missing). ES-MS for C₃₄H₁₀F₃NO₃S: 317.9 [M+H]⁺.

### Example 32

### 5-(4-Thiophen-2-yl-pyrimidin-2-ylsulfanylmethyl)-isoxazole-3-carboxylic acid

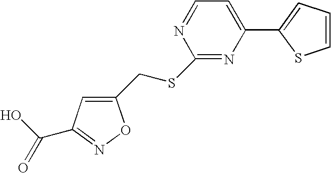

General Method B. Yield 97%. Pale yellow crystals. ¹H NMR (250 MHz, acetone-d₆) δ 8.61 (d, J=5.3 Hz, 1H), 8.02 (dd, J=3.8, 1.1 Hz, 1H), 7.79 (dd, J=5.0, 1.1 Hz, 1H), 7.62 (d, J=5.3 Hz, 1H), 7.24 (dd, J=5.0, 3.8 Hz, 1H), 6.76 (t, J=0.7 Hz, 1H), 4.69 (d, J=0.7 Hz, 2H). ES-MS for C₁₃H₉N₃O₃S₂: 319.9 [M+H]⁺.

### Example 33

### 5-(4-Toluenesulfonylmethyl)-isoxazole-3-carboxylic acid hydroxyamide

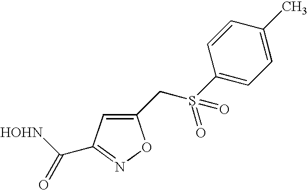

General Method C, Step 1. Yield 83%. White crystals. Mp 88-89° C. ¹H NMR (250 MHz, CDCl₃) δ 7.43 (d, J=8.3 Hz, 2H), 7.11 (d, J=8.3 Hz, 2H), 6.57 (s, 1H), 4.38 (s, 2H), 4.22 (q, J=7.1 Hz, 2H), 2.23 (s, 3H), 1.19 (q, J=7.1 Hz, 3H). ES-MS for C₁₄H₁₅NO₅S: 310.0 [M+H]+.

General Method C, Step 2. Yield 76%. White crystals. ¹H NMR (250 MHz, CD₃OD) δ 7.69(d, J=8.2 Hz, 2H), 7.42 (d, J=8.2 Hz, 2H), 6.64 (s, 1H), 4.90 (s, 2H), 2.45 (s, 3H). ¹³C NMR (250 MHz, CD₃OD) δ 164.6, 159.1, 158.8, 147.5, 136.7, 131.5, 129.9, 106.7, 22.0 (one signal missing). ES-MS for C₁₂H₁₂N₂O₅S: 296.9 [M+H]⁺.

### Example 34

### 5-(4-Chloro-benzenesulfonylmethyl)-isoxazole-3-carboxylic acid hydroxyamide

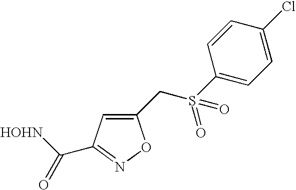

General Method C, Step 1. Yield 97%. White crystals. Mp 107-109° C. ¹H NMR (250 MHz, CDCl₃) δ 7.70 (d, J=8.7 Hz, 2H), 7.52 (d, J=8.7 Hz, 2H), 6.81 (s, 1H), 4.59 (s, 2H), 4.43 (q, J=7.1 Hz, 2H), 1.40 (t, J=7.1 Hz, 3H). ¹³C NMR (250 MHz, CDCl₃) δ 161.9, 159.1, 156.8, 141.6, 135.8, 129.9, 129.8, 106.7, 62.4, 53.8, 14.0. ES-MS for C₁₃H₁₂ClNO₅S: 329.8 [M+H]⁺.

General Method C, Step 2. Yield 84%. White crystals. ¹H NMR (250 MHz, CD₃OD) δ 7.80 (d, J=8.7 Hz, 2H), 7.65 (d, J=8.7 Hz, 2H), 6.69 (s, 1H), 4.98 (s, 2H). ES-MS for C₁₁H₉ClN₂O₅S: 316.8 [M+H]⁺.

### Example 35

### 5-(3-Chloro-benzenesulfonylmethyl)-isoxazole-3-carboxylic acid hydroxyamide

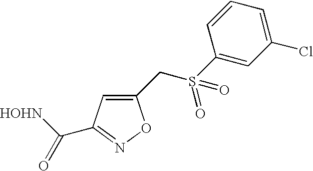

General Method C, Step 1. Yield 93%. White crystals. Mp 134-135° C. ¹H NMR (250 MHz, CDCl₃) δ 7.76 (m, 1H), 7.69 (m, 1H), 7.44 (m, 2H), 6.77 (d, J=0.7 Hz, 2H), 4.56 (d, J=0.7 Hz, 2H), 4.39 (q, J=7.1 Hz, 2H), 1.36 (t, J=7.1 Hz, 3H). ¹³C NMR (250 MHz, CDCl₃) δ 161.6, 159.1, 156.8, 139.1, 135.9, 134.8, 130.8, 128.3, 126.5, 106.8, 62.4, 53.8, 14.0. ES-MS for C₁₃H₁₂ClNO₅S: 329.8 [M+H]⁺.

General Method C, Step 2. Yield 70%. White crystals. ¹H NMR (250 MHz, CD₃OD) δ 7.86 (m, 1H), 7.76 (m, 2H), 7.60 (m, 1H), 6.70 (s, 1H), 5.01 (s, 2H). ES-MS for C₁₁H₉ClN₂O₅S: 316.8 [M+H]⁺.

### Example 36

### 5-(2-Isopropyl-benzenesulfonylmethyl)-isoxazole-3-carboxylic acid hydroxyamide

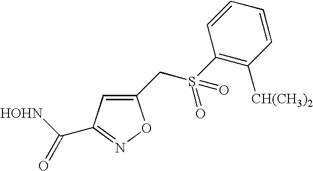

General Method C, Step 1. Yield 95%. Colourless oil. ¹H NMR (250 MHz, CDCl₃) δ 7.71 (dd, J=8.0, 1.4 Hz, 1H), 7.54 (m, 1H), 7.48 (m, 1H), 7.20 (m, 1H), 6.66 (s, 1H), 4.58 (s, 2H), 4.33 (q, J=7.1 Hz, 2H), 3.75 (h, J=6.8 Hz, 1H), 1.30 (t, J=7.1 Hz, 3H), 1.25 (d, J=6.8 Hz, 6H). ¹³C NMR (250 MHz, CDCl₃) δ 162.0, 159.0, 156.5, 149.6, 134.7, 134.4, 130.2, 128.0, 126.3, 106.3, 62.2, 53.8, 29.3, 24.0, 13.9. ES-MS for C₁₆H₁₉NO₅S: 338.0 [M+H]⁺.

General Method C, Step 2. Yield 62%. White crystals. Mp 170° C. (decompose). ¹H NMR (250 MHz, CD₃OD) δ 7.79 (m, 2H), 7.66 (m, 2H), 7.35 (m, 1H), 6.66 (s, 1H), 4.92 (s, 2H), 3.79 (h, J=6.8 Hz, 1H), 1.32 (s, J=6.8 Hz, 6H). ¹³C NMR (250 MHz, CD₃OD) δ. 164.0, 158.8, 151.4, 136.4, 136.0, 131.4, 129.4, 127.6, 106.4, 54.8, 30.7, 24.5 (one quaternary carbon missing). ES-MS for C₁₄H₁₆N₂O₅S: 324.9 [M+H]⁺.

### Example 37

### 5-(3-Methoxy-benzenesulfonylmethyl)-isoxazole-3-carboxylic acid hydroxyamide

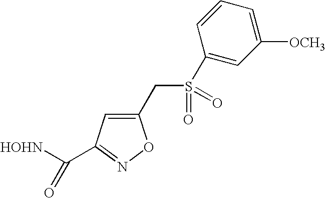

General Method C, Step 1. Yield 95%. White crystals. Mp 75-76° C. ¹H NMR (250 MHz, CDCl₃) δ 7.30 (m, 1H), 7.19 (m, 1H), 7.11 (m, 1H), 7.04 (ddd, J=8.1, 2.6, 1.1 Hz, 1H), 6.64 (s, 1H), 4.44 (s, 2H), 4.28 (q, J=7.1 Hz, 2H), 3.67 (s, 3H), 1.22 (t, J=7.1 Hz, 3H). ¹³C NMR (250 MHz, CDCl₃) δ 162.6, 160.5, 159.6, 157.2, 138.9, 131.0, 121.6, 120.8, 113.0, 107.0, 62.8, 56.1, 54.2, 14.4. ES-MS for C₁₄H₁₅NO₆S: 325.8 [M+H]⁺.

General Method C, Step 2. Yield 80%. White crystals. Mp 138° C. (decompose). ¹H NMR (250 MHz, CD₃OD) δ 7.54 (m, 1H), 7.39 (m, 1H), 7.30 (m, 2H), 6.66 (s, 1H), 4.94 (s, 2H), 3.84 (s, 3H). ¹³C NMR (250 MHz, CD₃OD) δ 164.2, 161.6, 158.8, 158.4, 140.4, 131.7, 121.9, 121.5, 114.0, 106.5, 56.3, 54.0. ES-MS for C₁₂H₁₂N₂O₆S: 312.8 [M+H]⁺.

### Example 38

### 5-(4-Toluenesulfinylmethyl)-isoxazole-3-carboxylic acid hydroxyamide

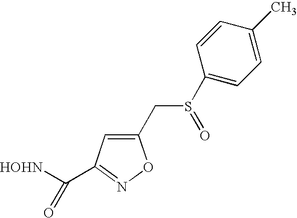

General Method D, Step 1. Yield 87%. Colourless crystals. Mp 77-79° C. ¹H NMR (250 MHz, CDCl₃) δ 7.16 (m, 2H), 7.07 (m, 2H), 6.38 (s, 1H), 4.20 (q, J=7.1 Hz, 2H), 4.05 (d, J=14.4 Hz, 1H), 3.95 (d, J=14.4 Hz, 1H), 2.18 (s, 3H), 1.18 (t, J=7.1 Hz, 3H). ¹³C NMR (250 MHz, CDCl₃) δ 163.3, 159.3, 156.4, 142.5, 138.5, 130.0, 123.8, 105.8, 62.1, 53.3, 21.3, 13.9. ES-MS for C₁₄H₁₅NO₄S: 294.0 [M+H]⁺.

General Method D, Step 2. Yield 79%. Pale pink crystals. Mp 160° C. (decompose). ¹H NMR (250 MHz, CD₃OD) δ 7.45 (d, J=8.2 Hz, 2H), 7.37 (d, J=8.2 Hz, 2H), 6.50 (s, 1H), 4.55 (d, J=14.1 Hz, 1H), 4.41 (d, J=14.1 Hz, 1H), 2.41 (s, 3H). ¹³C NMR (250 MHz, CD₃OD) δ 165.0, 158.6, 144.2, 139.4, 131.2, 125.4, 105.8, 53.2, 21.4 (one signal missing). ES-MS for C₁₂H₁₂N₂O₄S: 280.9 [M+H]⁺.

### Example 39

### 5-[1-(4-Toluenesulfonyl)-ethyl]-isoxazole-3-carboxylic acid hydroxyamide

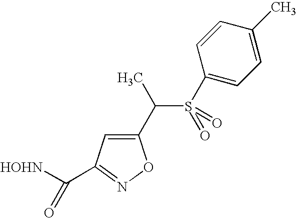

General Method E, Step 1, by using methyliodide (1.5 mmol) as electrophile. White crystals. Mp 75-77° C. ¹H NMR (250 MHz, CDCl₃) δ 7.58 (d, J=8.5 Hz, 2H), 7.32 (d, J=8.5 Hz, 2H), 6.77 (s, 1H), 4.57(q, J=7.2 Hz, 1H), 4.45 (q, J=7.1 Hz, 2H), 2.45 (s, 3H), 1.78 (d, J=7.2 Hz, 3H), 1.42 (t, J=7.1 Hz, 3H). ¹³C NMR (250 MHz, CDCl₃) δ 167.6, 159.3, 156.5, 145.7, 132.9, 129.9, 129.0, 105.3, 62.3, 58.7, 21.6, 14.0, 12.8. ES-MS for C₁₇H₂₁NO₅S: 351.9 [M+H]⁺.

General Method E, Step 2. Yield (after flash column chromatography) 69%. White sticky foam. ¹H NMR (250 MHz, CD₃OD) δ 7.61 (m, 2H), 7.46 (m, 2H), 6.66 (s, 1H), 4.93 (q, J=7.2 Hz, 1H), 2.45 (s, 3H), 1.70 (d, J=7.2 Hz, 3H). ¹³C NMR (250 MHz, CD₃OD) δ 168.7, 158.5, 158.4, 147.2, 134.6, 131.0, 130.2, 105.3, 59.5, 21.5, 12.9. ES-MS for C₁₃H₁₄N₂O₅S: 310.9 [M+H].

### Example 40

### 5-[1-(4-Chloro-benzenesulfonyl)ethyl]-isoxazole-3-carboxylic acid hydroxyamide

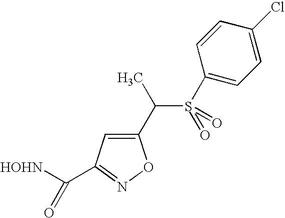

General Method E, Step 1, by using methyliodide as electrophile (1.5 mmol). Yield (after FC) 46%. Colourless crystals. Mp 114-115° C. ¹H NMR (250 MHz, CDCl₃) δ 7.62 (m, 2H), 7.49 (m, 2H), 6.77 (d, J=0.5 Hz, 1H), 4.57 (dq, J=7.2, 0.5 Hz, 1H), 4.43 (q, J=7.1 Hz, 2H), 1,77 (d, J=7.2 Hz, 3H), 1.04 (t, J=7.1 Hz, 3H). ¹³C NMR (250 MHz, CDCl₃) δ 167.1, 159.2, 156.6, 141.5, 134.4, 130.4, 129.7, 105.5, 62.4, 58.7, 14.0, 12.6. ES-MS for C₁₄H₁₄ClNO₅S: 347.8 [M+H]⁺.

General Method E, Step 2. White crystals. Mp 162° C. (decompose) ¹H NMR (250 MHz, CD₃OD) δ 7.72 (m, 2H), 7.62 (m, 2H), 6.72 (s, 1H), 5.01 (q, J=7.2 Hz, 1H), 1.73 (d, J=7.2 Hz, 3H). ES-MS for C₁₂H₁₁ClN₂O₅S: 227.9 [M+H]⁺.

### Example 41

### 5-[1-(3-Methoxy-benzenesulfonyl)-propyl]-isoxazole-3-carboxylic acid hydroxyamide

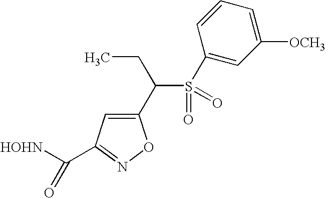

General Method E, Step 1, by using ethylbromide as electrophile (1.5 mmol). Yield (after flash column chromatography) 30%. Colourless oil. ¹H NMR (250 MHz, CDCl₃) δ 7.34 (dd, J=8.8, 7.7 Hz, 1H), 7.19 (m, 2H), 7.10 (m, 2H), 6.71 (s, 1H), 4.37 (q, J=7.1 Hz, 2H), 4.30 (dd, J=11.5, 3.9 Hz, 1H), 3.73 (s, 3H), 2.37 (ddq, J=13.9, 7.5, 3.9 Hz, 1H), 2.05 (ddq, J=13.7, 11.5, 7.3 Hz, 1H), 1.35 (t, J=7.1 Hz, 3H), 0.89 (t, J=7.5 Hz, 3H). ¹³C NMR (250 MHz, CDCl₃) δ 166.7, 159.9, 159.3, 156.6, 137.6, 130.3, 121.1, 121.0, 113.1, 105.7, 65.4, 62.3, 55.6, 21.0, 14.0, 11.2. ES-MS for C₁₆H₁₉NO₆S: 354.1 [M+H]⁺.

General Method E, Step 2. Yield 65%. Pink foam that slowly crystallized. ¹H NMR (250 MHz, CD₃OD) δ 7.48 (t, J=7.9 Hz, 1H), 7.28 (m, 2H), 7.18 (m, 1H), 6.70 (s, 1H), 4.84 (dd, J=11.2, 4.0 Hz, 1H), 3.82 (s, 3H), 2.34 (ddq, J=13.8, 7.8, 4.0 Hz, 1H), 2.12 (ddq, J=13.8, 11.2, 7.8 Hz, 1H), 0.95 (t, J=7.8 Hz, 3H). ¹³C NMR (250 MHz, CD₃OD) δ 167.7, 161.5, 158.6, 158.3, 139.3, 131.5, 122.1, 121.9, 114.4, 106.2, 65.8, 56.2, 21.8, 11.3. ES-MS for C₁₄H₁₆N₂O₆S: 341.1 [M+H]⁺.

### Example 42

### 5-[1-(2-Isopropyl-benzenesulfonyl)-propyl]-isoxazole-3-carboxylic acid hydroxyamide

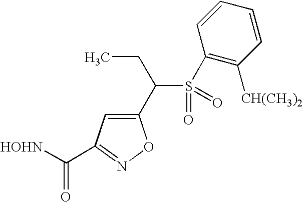

General Method E, Step 1, by using ethylbromide as electrophile (1.5 mmol). Yield (after flash column chromatography) 42%. Colourless oil. ¹H NMR (250 MHz, CDCl₃) δ 7.70 (dd, J=8.1, 1.4 Hz, 1H), 7.53 (m, 2H), 7.23 (m, 1H), 6.75 (s, 1H), 4.41 (q, J=7.1 Hz, 2H), 4.40 (dd, superposed, 1H), 3.80 (h, J=6.8 Hz, 1H), 2.43 (ddq, J=13.8, 7.5, 3.9 Hz, 1H), 2.19 (ddq, J=13.8, 11.2, 7.3 Hz, 1H), 1.39 (t, J=7.1 Hz, 3H), 1.36 (d, J=6.8 Hz, 3H), 1.28 (d, J=6.7 Hz, 3H), 0.97 (t, J=7.5 Hz, 3H). ¹³C NMR (250 MHz, CDCl₃) δ 166.9, 159.3, 156.5, 150.0, 134.6, 133.8, 130.8, 128.1, 126.2, 105.2, 65.3, 62.3, 29.5, 24.5, 23.9, 20.9, 14.0, 11.3. ES-MS for C₁₈H₂₃NO₅S: 366.1 [M+H]⁺.

General Method E, Step 2. Yield 74%. Pink foam that slowly crystallized. ¹H NMR (250 MHz, CD₃OD) δ 7.73 (m, 1H), 7.62 (m, 2H), 7.32 (m, 1H), 6.71 (s, 1H), 4.69 (dd, J=11.0, 4.3 Hz, 1H), 3.76 (h, J=6.7 Hz, 1H), 2.36 (m, 1H), 2.22 (m, 1H), 1.33 (d. J=6.7 Hz, 3H), 1.25 (d, J=6.7 Hz, 3H), 0.97 (t, J=7.4 Hz, 3H). ¹³C NMR (250 MHz, CD₃OD) δ 167.6, 158.6, 158.2, 151.6, 135.9, 135.5, 132.0, 129.3, 127.5, 105.9, 66.4, 30.6, 24.8, 24.3, 21.7, 11.4. ES-MS for C₁₆H₂₀N₂O₅S: 353.1 [M+H]⁺.

Biological Assays

The compounds of this invention may be tested in the following biological assay in order to determine the concentration of compound (IC₅₀) required for exhibiting the desired pharmacological effect.

To find inhibitors of Peptide Deformylase (PDF), a colorimetric cell free assay for measuring the enzymatic activity of PDF has been adapted to the microtiter plate format (96 wells). The assay comprises three components, purified PDF, f-Met-Ala as substrate and TNBS as the detecting agent of primary amino groups. The resulting TNP-NH-Met-Ala sulfite complex can be detected at 420 nm. PDF enzymes, containing Fe²⁺ as the native metal, are purified and are stabilized by the addition of tris(2-carboxyethyl)phosphine (TCEP).

Chemicals

| | |
|---|---|
| Boric acid | (Fluka, cat no. 15663) |
| BSA | (Fluka, cat no. 5476), Bovine serum albumin |
| Catalase | (Fluka, cat no. 60640) |
| DMSO | dimethylsulfoxide |
| f-Met-Ala | (Bachem, cat no. G-1855) |
| Methanol | (Fluka, cat no. 65544) |
| MOPS | (Fluka, cat no. 69947), |
| | 4-Morpholinepropanesulfonic acid hemisodium salt, |
| NaCl | (Fluka, cat no. 71382) |
| NaH₂PO₄ | (Fluka, cat no. 71505) |
| NaOH | (Fluka, cat no. 71689) |
| Na₂SO₃ | (Fluka, cat no. 71988) |
| Sodium 4- | (Fluka, cat no. 55540) |
| (hydroxymercurio) | |
| benzoate | |
| TNBS | (Fluka, cat no. 92823), |
| | 2,4,6-trinitrobenzene sulfonic acid. |

Bacterial Peptide Deformylase (PDF) Assay

The IC₅₀ value of a compound of the invention as a bacterial PDF inhibitor was determined using the following assay.

Materials:

Assay buffer: 0.1M MOPS pH was adjusted to 7.2 with NaOH, containing 0.25 M NaCl, 100 μg/mL catalase and 1 mg/mL BSA.

Enzyme Mix:

E. coli enzyme (2.5 mg/ml) 10 μl+290 μl Assay buffer, 1 μl per ml enzyme mix.

S. aureus (15 mg/ml) 10 μl+990 μl Assay buffer, 0.3 μl per ml enzyme mix.

Substrate mix: 10 mM f-Met-Ala was made up from 200 mM f-Met-Ala in methanol with assay buffer.

TNBS solution: Freshly dilute 1 M TNBS stock solution diluted 1:10 with water.

Buffer C, 0.5 M borate buffer adjusted to pH 9.5 with NaOH.

Buffer D: 0.2 ml of freshly prepared 0.5 M Na₂SO₃ was mixed with 9.8 mL of 0.5M NaH₂PO₄.

Inhibitor solution: 2 mM Sodium 4-(hydroxymercurio) benzoate in assay buffer.

Compound mix: Compound of formula I dissolved in DMSO in a 10 mg/mL stock solution. Further dilutions were made in DMSO in the concentration range between 0.05 to 100 mM.

Method (Assay Conditions):

The assay was performed in a 96 Microtiter plate containing test compound. To each well containing test compound mix was added 75 microliter of enzyme mix followed by the addition of 25 microliter of substrate mix. The resulting mix was incubated for 30 minutes at room temperature with shaking. TNBS solution (50 microliter/well) was added and the resulting mixture was incubated for 15 minutes under shaking. Buffer C was then added (20 microliter/well). After incubating at room temperature for 15 minutes under shaking, buffer D was added (50 microliter/well). The optical diffraction was then measured at 420 nm, thereby determining the IC₅₀ value.

Results:

PDF enzyme inhibitory activity

IC₅₀, (μM)^{a}

| | E. coli, | S. aureus, |
|---|---|---|
| Compound | PDF enzyme | PDF enzyme |
| Example 1 | 20.0 | 4.0 |
| Example 2 | 14.5 | 1.3 |
| Example 3 | 9.8 | 2.3 |
| Example 4 | 62.5 | 3.8 |
| Example 5 | 4.4 | 2.0 |
| Example 6 | 60.0 | 4.9 |
| Example 7 | 7.0 | 2.0 |
| Example 8 | 14.0 | 7.6 |
| Example 9 | 29.5 | 37.7 |
| Example 10 | 31.3 | 24.0 |
| Example 11 | 6.5 | 2.2 |
| Example 12 | 20.7 | 1.5 |
| Example 13 | 22.0 | 8.7 |
| Example 14 | 25.3 | 9.3 |
| Example 16 | 3.4 | 12.7 |
| Example 17 | 7.6 | 2.2 |
| Example 33 | 11.5 | 1.4 |
| Example 34 | 7.5 | 0.8 |
| Example 35 | 6.8 | 1.7 |
| Example 36 | 10.8 | 3.4 |
| Example 37 | 26.3 | 6.5 |
| Example 38 | 12.0 | 0.9 |

^{a}values are means of three experiments.

The results show that the compounds tested have peptide deformylase inhibitory activity.

The above specification and Examples fully disclose how to make and use the compounds of the present invention. However, the present invention is not limited to the particular embodiments described hereinabove, but includes all modifications thereof within the scope of the following claims. The various references to journals, patents and other publications which are cited herein comprise the state of the art and are incorporated herein by reference as though fully set forth.

### LITERATURE LIST

Adams, J. M.; Capecchi, M. R. Proc. Natl. Acad. Sci., USA, 1966, 55, 147-155.
Adams, J. M. J. Mol. Biol. 1968, 33, 571-589.
Clements, J. M.; Ayscough, A. P.; Keavey, K.; East, S. P. Curr. Med. Chem. Anti-Infective Agents 2002, 1, 239-249.
Gennaro, A. R.; Gennaro A. L. Remington, The Science and Practice of Pharmacy, 19th ed., Mack Publishing Co., Easton, Pa., 1995.
Giglione, C.; Pierre, M.; Meinnel, T. Mol. Microbiol. 2000, 36, 1197-1205.
Giglione, C.; Meinnel, T. Emerg. Ther. Targets 2001, 5, 41-57.
Green, T. W. Protective Groups In Organic Synthesis, John Wiley & Sons, New York, 1981.
Hanessian, S.; Mackay, D. B.; Moitessier, N. J. Med. Chem. 2001, 44, 3074-3082
Hauser, C. R.; Renfrow, W. B. Jr. Org. Synth. Coll. 1943, Coll. Vol. II, p. 67-67.
Micetich, R. G.; Shaw, C. C.; Hall, T. W.; Spevak, P.; Fortier, R. A.; Wolfert, P.; Foster, B. C.; Bains, B. K. Heterocycles, 1985, 23, 571-583
Pei, D. Emerg. Ther. Targets 2001, 5, 23-40.
Yuan, Z.; Trias, J.; White, R. J. Drug Discov. Today 2001, 6, 954-961.
J. Pharm. Sci. 1977, 66, 2.

1. A compound of formula (I) 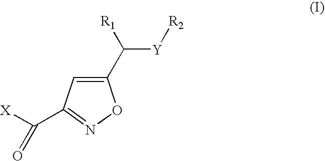
or a pharmaceutically acceptable salt or ester thereof, wherein
X is selected from hydroxy, C₁₋₆ alkoxy, and —NH—OH;
Y is selected from S, SO, SO₂ and O;
R₁ is selected from hydrogen, C₁₋₆ alkyl, C₂₋₆ alkenyl, C₃₋₁₀ cycloalkyl, C₃₋₇ heterocycloalkyl, an unsubstituted or substituted C₁₋₆ alkylaryl group, an unsubstituted or substituted C₁₋₆ alkylheteroaryl group, an unsubstituted or substituted aryl group, an unsubstituted or substituted heteroaryl group, wherein a substituted group in connection with R₁ is substituted with one, two, three or four substituents independently selected from the group consisting of halogen, C₁₋₆ alkyl and C₁₋₆ alkoxy;
and R₂ is selected from C₁₋₆ alkyl, C₃₋₁₀ cycloalkyl, C₃₋₇ heterocycloalkyl, an unsubstituted or substituted aryl group, an unsubstituted or substituted heteroaryl group, an unsubstituted or substituted C₁₋₆ alkylaryl group, or an unsubstituted or substituted C₁₋₆ alkylheteroaryl group, wherein a substituted group in connection with R₂ is substituted with one, two, three or four substituents independently selected from the group consisting of halogen, C₁₋₆ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano;
with the proviso that when X is hydroxy or ethoxy, Y is S, SO, or SO₂ and R₁ is hydrogen, R₂ cannot be methyl;
with the proviso that when X is methoxy or ethoxy, Y is S and R₁ is hydrogen, R₂ cannot be a 4-halogen-pyridazin-3-on;
with the proviso that when X is methoxy, Y is S and R₁ is methyl, R₂ cannot be a 4-halogen-pyridazin-3-on.

2. A compound according to claim 1, wherein X is —NH—OH.

3. A compound according to claim 1, wherein X is hydroxy.

4. A compound according to claim 1, wherein X is selected from the group consisting of methoxy, ethoxy and propoxy.

5. A compound according to claim 1, wherein Y is selected from S, SO and SO₂.

6. A compound according to claim 1, wherein Y is S.

7. A compound according to claim 1, wherein R₁ is selected from the group consisting of hydrogen, C₁₋₆ alkyl, and C₃₋₁₀ cycloalkyl.

8. A compound according to claim 1, wherein R₁ is selected from the group consisting of hydrogen, methyl, ethyl, n-propyl, isopropyl, n-butyl, sec-butyl, isobutyl, tert-butyl, pentyl, cyclopentyl and cyclohexyl.

9. A compound according to claim 1, wherein R₁ is an unsubstituted or substituted aryl group.

10. A compound according to claim 1, wherein R₁ is an unsubstituted or substituted heteroaryl group.

11. A compound according to claim 1, wherein R₁ is selected from an unsubstituted or substituted C₁₋₆ alkylaryl group and an unsubstituted or substituted C₁₋₆ alkylheteroaryl group.

12. A compound according to claim 1, wherein R₂ is selected from an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group.

13. A compound according to claim 1, wherein R₂ is an unsubstituted or substituted aryl group.

14. A compound according to claim 1, wherein R₂ is an unsubstituted or substituted phenyl group.

15. A compound according to claim 1, wherein R₂ is a substituted phenyl group, wherein a substituted phenyl is substituted with one, two, three or four substituents independently selected from methyl, ethyl, n-propyl, butyl, isopropyl, isobutyl, sec-butyl, tert-butyl, fluoro, chloro, bromo, iodo, methoxy, trifluoromethyl, trifluoromethoxy, aminocarbonylmethyl and thiophenyl.

16. A compound according to claim 1, wherein R₂ is a substituted phenyl group, substituted with one, two, three or four substituents independently selected from chloro, bromo, trifluoromethyl or trifluoromethoxy.

17. A compound according to claim 1, wherein R₂ is an unsubstituted or substituted group, wherein the group is selected from biphenyl, diphenyl, naphtyl, benzothiophenylmethyl, thiophenyl-pyrimidinyl, pyridyl, quinolyl, pyrimidinyl, pyrazinyl, pyridazinyl, pyrazolyl, imidazolyl, triazolyl, tetrazolyl, oxazolyl, isoxazolyl, thiazolyl, isothiazolyl, thiophenyl, furanyl, thiadiazolyl and oxadiazolyl.

18. A compound according to claim 1, wherein R₂ is selected from C₁₋₆ alkyl, C₃₋₁₀ cycloalkyl and C₃₋₇ heterocycloalkyl.

19. A compound according to claim 1 selected from the R- and the S-stereoisomers, if any, of:
5-(4-toluenesulfanylmethyl)-isoxazole-3-carboxylic acid hydroxyamide;
5-(4-chloro-phenylsulfanylmethyl)-isoxazole-3-carboxylic acid hydroxyamide;
5-(3-chloro-phenylsulfanylmethyl)-isoxazole-3-carboxylic acid hydroxyamide;
5-(2-chloro-phenylsulfanylmethyl)-isoxazole-3-carboxylic acid hydroxyamide;
5-(3,4-dichloro-phenylsulfanylmethyl)-isoxazole-3-carboxylic acid hydroxyamide;
5-(2-bromo-phenylsulfanylmethyl)-isoxazole-3-carboxylic acid hydroxyamide;
5-(3-bromo-phenylsulfanylmethyl)-isoxazole-3-carboxylic acid hydroxyamide;
5-(2-isopropyl-phenylsulfanylmethyl)-isoxazole-3-carboxylic acid hydroxyamide;
5-(2,4,6-trimethyl-phenylsulfanylmethyl)-isoxazole-3-carboxylic acid hydroxyamide;
5-(3-methoxy-phenylsulfanylmethyl)-isoxazole-3-carboxylic acid hydroxyamide;
5-(4-fluoro-phenylsulfanylmethyl)-isoxazole-3-carboxylic acid hydroxyamide;
5-(4-trifluoromethyl-phenylsulfanylmethyl)-isoxazole-3-carboxylic acid hydroxyamide;
5-(2-trifluoromethyl-phenylsulfanylmethyl)-isoxazole-3-carboxylic acid hydroxyamide;
5-(2-trifluoromethoxy-phenylsulfanylmethyl)-isoxazole-3-carboxylic acid hydroxyamide;
5-(3-trifluoromethyl-benzylsulfanylmethyl)-isoxazole-3-carboxylic acid hydroxyamide;
5-(4-toluenesulfanylmethyl)-isoxazole-3-carboxylic acid;
5-(4-chloro-phenylsulfanylmethyl)-isoxazole-3-carboxylic acid;
5-(3-chloro-phenylsulfanylmethyl)-isoxazole-3-carboxylic acid;
5-(2-isopropyl-phenylsulfanylmethyl)-isoxazole-3-carboxylic acid;
5-(2,4,6-trimethyl-phenylsulfanylmethyl)-isoxazole-3-carboxylic acid;
5-(4-methoxy-phenylsulfanylmethyl)-isoxazole-3-carboxylic acid;
5-(3-methoxy-phenylsulfanylmethyl)-isoxazole-3-carboxylic acid;
5-(4-fluoro-phenylsulfanylmethyl)-isoxazole-3-carboxylic acid;
5-(4-trifluoromethyl-phenylsulfanylmethyl)-isoxazole-3-carboxylic acid;
5-(2-trifluoromethyl-phenylsulfanylmethyl)-isoxazole-3-carboxylic acid;
5-(2-trifluoromethoxy-phenylsulfanylmethyl)-isoxazole-3-carboxylic acid;
5-(3-trifluoromethyl-benzylsulfanylmethyl)-isoxazole-3-carboxylic acid;
5-(4-toluenesulfonylmethyl)-isoxazole-3-carboxylic acid hydroxyamide;
5-(4-chloro-benzenesulfonylmethyl)-isoxazole-3-carboxylic acid hydroxyamide;
5-(3-chloro-benzenesulfonylmethyl)-isoxazole-3-carboxylic acid hydroxyamide;
5-(2-isopropyl-benzenesulfonylmethyl)-isoxazole-3-carboxylic acid hydroxyamide;
5-(3-methoxy-benzenesulfonylmethyl)-isoxazole-3-carboxylic acid hydroxyamide;
5-(4-toluenesulfinylmethyl)-isoxazole-3-carboxylic acid hydroxyamide;
5-[1-(4-toluenesulfonyl)-ethyl]-isoxazole-3-carboxylic acid hydroxyamide;
5-[1-(4-chloro-benzenesulfonyl)-ethyl]-isoxazole-3-carboxylic acid hydroxyamide;
5-[1-(3-methoxy-benzenesulfonyl)-propyl]-isoxazole-3-carboxylic acid hydroxyamide;
5-[1-(2-isopropyl-benzenesulfonyl)-propyl]-isoxazole-3-carboxylic acid hydroxyamide;
5-(benzothiophen-2-ylmethylsulfanylmethyl)-isoxazole-3-carboxylic acid hydroxyamide;
5-(4-thiophen-2-yl-pyrimidin-2-ylsulfanylmethyl)-isoxazole-3-carboxylic acid hydroxyamide;
5-(benzothiophen-2-ylmethylsulfanylmethyl)-isoxazole-3-carboxylic acid; and
5-(4-thiophen-2-yl-pyrimidin-2-ylsulfanylmethyl)-isoxazole-3-carboxylic acid.

20. A compound according to, which in the PDF assay exhibits an IC₅₀ value of less than 500 μM.

21-24. (canceled)

25. A pharmaceutical composition comprising, as an active substance, a compound as defined in claim 1 or a pharmaceutical acceptable salt thereof together with a pharmaceutical acceptable carrier or diluent.

26. A pharmaceutical composition according to claim 25 comprising a second active substance having antibacterial activity.

27. A pharmaceutical composition according to claim 25, comprising from about 1 μg to about 1000 mg of the active substance or a pharmaceutical acceptable salt or ester thereof.

28. A pharmaceutical composition according to in unit dosage form.

29-30. (canceled)

31. A pharmaceutical composition according to claim 25 for oral, nasal, transdermal, pulmonal or parenteral administration.

32. A method for the treatment of one or more ailments, the method comprising administering to a subject in need thereof an effective amount of a compound of formula (I) 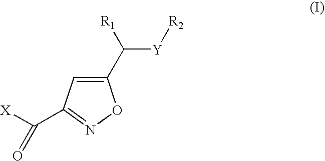
or a pharmaceutically acceptable salt or ester thereof, wherein
X is selected from hydroxy, C₁₋₆ alkoxy, and —NH—OH;
Y is selected from S, SO, SO₂ and O;
R₁ is selected from hydrogen, C₁₋₆ alkyl, C₂₋₆ alkenyl, C₃₋₁₀ cycloalkyl, C₃₋₇ heterocycloalkyl, an unsubstituted or substituted C₁₋₆ alkylaryl group, an unsubstituted or substituted C₁₋₆ alkylheteroaryl group, an unsubstituted or substituted aryl group, an unsubstituted or substituted heteroaryl group, wherein a substituted group in connection with R₁ is substituted with one, two, three or four substituents independently selected from the group consisting of halogen, C₁₋₆ alkyl and C₁₋₆ alkoxy;
and R₂ is selected from C₁₋₆ alkyl, C₃₋₁₀ cycloalkyl, C₃₋₇ heterocycloalkyl, an unsubstituted or substituted aryl group, an unsubstituted or substituted heteroaryl group, an unsubstituted or substituted C₁₋₆ alkylaryl group, or an unsubstituted or substituted C₁₋₆ alkylheteroaryl group, wherein a substituted group in connection with R₂ is substituted with one, two, three or four substituents independently selected from the group consisting of halogen, C₁₋₆ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano.

33. (canceled)

34. A method according to claim 32, wherein the compound in the PDF assay exhibits an IC50 value of less than 500 μM.

35. A method according to claim 32, wherein the effective amount of the compound is in a range of from about 1 μg to about 1000 mg per day.

36-40. (canceled)

41. A method for the treatment of a patient suffering from or susceptible to a bacterial infection, the method comprising administering to the patient an effective amount of a compound of formula (I) 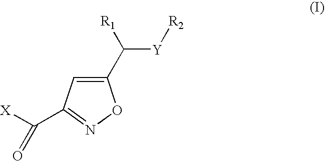
or a pharmaceutically acceptable salt or ester thereof, wherein
X is selected from hydroxy, C₁₋₆ alkoxy, and —NH—OH;
Y is selected from S, SO, SO₂ and O;
R₁ is selected from hydrogen, C₁₋₆ alkyl, C₂₋₆ alkenyl, C₃₋₁₀ cycloalkyl, C₃₋₇ heterocycloalkyl, an unsubstituted or substituted C₁₋₆ alkylaryl group, an unsubstituted or substituted C₁₋₆ alkylheteroaryl group, an unsubstituted or substituted aryl group, an unsubstituted or substituted heteroaryl group, wherein a substituted group in connection with R₁ is substituted with one, two, three or four substituents independently selected from the group consisting of halogen, C₁₋₆ alkyl and C₁₋₆ alkoxy;
and R₂ is selected from C₁₋₆ alkyl, C₃₋₁₀ cycloalkyl, C₃₋₇ heterocycloalkyl, an unsubstituted or substituted aryl group, an unsubstituted or substituted heteroaryl group, an unsubstituted or substituted C₁₋₆ alkylaryl group, or an unsubstituted or substituted C₁₋₆ alkylheteroaryl group, wherein a substituted group in connection with R₂ is substituted with one, two, three or four substituents independently selected from the group consisting of halogen, C₁₋₆ alkyl, C₃₋₁₀ cycloalkyl, C₁₋₆ alkoxy, hydroxy, aminocarbonylC₁₋₆ alkyl, trifluoromethyl, trifluoromethoxy, trifluoromethylthio, heteroaryl, nitro and cyano.

42. The method of claim 41 wherein the patient is suffering from a bacterial infection.

43. The method of claim 41 wherein the patient has been identified and selected for treatment as suffering from a bacterial infection and the compound is administered to the selected patient.

44. The method of claim 41 wherein the patient is suffering from an infection associated with an organism belonging to the group consisting of Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, Helicobacter, Campylobacter, Legionella and Neisseria.

46. The method of claim 41 wherein the patient is suffering from an infection associated with an organism belonging to the group consisting of Staphylococcus aureus, Staphylococcus epidermidis, Enterococcus faecium, Enterococcus faecalis, Streptococcus pneumoniae, Haemophilus influenza, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae.
